(19) United States
(12) Patent Application Publication
(10) Pub. No.: US 2025/0255590 A1
(43) Pub. Date: Aug. 14, 2025

(54) DEVICE AND METHOD FOR COLLECTING BIOLOGICAL SAMPLES FROM A BEAM

(71) Applicant: UC-CARE LTD., Yokneam (IL)

(72) Inventors: Yeshayahu SCHATZBERGER, Haifa (IL); Ichai PEREZ, Haifa (IL); Keren SCHWEITZER, Tal-El (IL); Tomer SCHATZBERGER, Tampa, FL (US)

(21) Appl. No.: 18/867,127

(22) PCT Filed: May 22, 2023
(86) PCT No.: PCT/IB2023/055228

§ 371 Date: Nov. 19, 2024

Related U.S. Application Data
(60) Provisional application No. 63/344,662, filed on May 23, 2022.

(51) Int. Cl. A61B 10/02

(52) U.S. Cl. CPC A61B 10/0275

(57) ABSTRACT

A device for collecting a biological sample from a biopsy needle includes a housing that can be used to support a biopsy needle, and a cassette that has an adhering surface. The collecting of the biological sample from a biopsy needle includes engaging the adhering surface of the cassette with the biopsy needle and after that urging relative motion between the biopsy needle and the adhering surface in order to assist in collecting the biological sample from the biopsy needle.

### FIELD OF THE INVENTION

The invention, in some embodiments, relates to the field of handling biological samples, and more particularly, but not exclusively, to devices and methods intended for collecting a biological sample from a shaft or from a biopsy needle.

### BACKGROUND OF THE INVENTION

Core biopsy is a routine procedure used to obtain a sample of a biological tissue from a live organ for a laboratory examination. FIGS. 1A-1D depict schematically a core biopsy needle 10 used for taking core biopsy samples. Core biopsy needle 10 comprises a mandrel 12 and a cannula 14. Mandrel 12 is an elongated solid beam having a form of a needle with a pointed end at a distal tip 16 of needle 10 and a notch 18 adjacent to distal tip 16. Notch 18 is an interior shaft recess, used for receiving the tissue sample. Cannula 14 is a sleeve exterior to mandrel 12 and is configured for sliding over mandrel 12.

During a core biopsy procedure, distal tip 16 of core biopsy needle 10 is brought up to a few millimeters from a region to be sampled in an organ as is schematically depicted in FIG. 1A. Mandrel 12 is advanced forward into the organ, typically at a high speed to reduce pain in an awakened patient, allowing organ tissue to fill in, at least partially, notch 18 as is schematically depicted in FIG. 1B. Cannula 14 is then advanced forward over the mandrel, thereby cutting off a sample tissue which is left in notch 18 as is schematically depicted in FIG. 1C. Core biopsy needle 10 is then removed from the organ and Cannula 14 is pulled back in order to expose the sample tissue on notch 18 as is schematically depicted in FIG. 1D.

International Patent Application PCT/IL2013/050031, publication number WO/2013/105095, filed on Jan. 10, 2013, which is incorporated herein by reference and referred to as Application '031, discloses a device for collecting onto a cassette a biological tissue carried on a shaft. The device comprises a base, a cassette holder and a needle bed. The needle bed is attached to one of the base and the cassette holder and is configured to support, substantially in a pre-defined position, a shaft carrying a biological tissue. The other one of the base and the cassette holder is configured to support a cassette attached thereto. The cassette holder is movable between positions relative to the base, thereby a cassette suitably attached to the device is movable relative to the needle bed, so that in a first position the cassette and the needle bed are distant from one another, and in a second position the cassette and the needle bed are situated proximal to one another having a predefined arrangement relative to one another.

According to some embodiments of Application '031, the device may be equipped with a cassette having an adhering surface for collecting and holding a biological tissue on the sample sheet. In some embodiments the cassette comprises a cassette base comprising a base slab. The cassette may be assembled with a sample sheet having an adhering surface configured to adhere to a biological tissue, whereas at least a portion of the adhering surface is accessible for attaching to a biological tissue.

Application '031 further discloses, according to an aspect of some embodiments, a method for collecting a core biopsy sample from a core biopsy needle. According to some embodiments, the method allows attaching a core biopsy sample onto a cassette so that the position and orientation of the sample tissue on the notch of the core biopsy needle is known and preserved.

As referred to herein, the expressions “biological tissue”, “biological sample” and “biological specimen” or simply “tissue” or “sample” may be used interchangeably, referring to continuous tissue, solid but typically not dry, typically but not necessarily soft, typically but not necessarily macroscopic in size, obtained as a single piece from a live organ. A biopsy sample, obtained e.g. by a core biopsy procedure, is an example of a biological sample as referred to herein. As referred to herein, a beam configured to carry a biological tissue is an elongated rigid object having a long axis defined along its length. A beam further comprises a flat surface suitable for supporting a biological tissue and typically but not necessarily comprises a handle configured for holding the beam e.g. by hand, being separated from the tissue-carrying surface along the beam length. A scalpel and a biopsy needle are non-limiting examples of beams configured to carry and support a biological tissue thereon.

### SUMMARY OF THE INVENTION

Aspects of the disclosure, in some embodiments thereof, relate to handling biological samples. More specifically, aspects of the disclosure, in some embodiments thereof, relate to devices and methods intended for collecting onto a cassette a biological sample from a shaft or from a biopsy needle.

As described above, known methods and devices for collecting a biological sample from a shaft or a core biopsy needle onto a cassette, involve nearing the shaft with the biological sample to the cassette by moving the cassette holder from a first position wherein the shaft and the cassette are distant, to a second position wherein the shaft and the cassette are proximal to one another. According to some known methods and devices, the biological sample may be attached to the cassette upon contacting the cassette. According to some known methods and devices, the cassette may comprise an adhering surface, configured to adhere to a biological sample upon contacting the biological sample. Thus, when contact is formed between the biological sample and the cassette or an adhering surface thereof, the biological sample adheres to the cassette, and when the cassette holder is moved to the first position, distant from the shaft, the biological sample remains attached, or stuck, to the cassette while being detached from the shaft.

The force or forces between a shaft and a biological sample supported thereon, may be complex and may be different for different biological samples supported on a same shaft or on a same core biopsy needle. Such force or forces may be different for a same biological sample supported on different shafts, and may be different even for different points of contact or regions of contact, of the biological sample and the supporting surface of the shaft or of the core biopsy needle (such as the notch). In other words, although generally a biological sample may adhere better (that is to say, by stronger forces) to the cassette than to the shaft, in some instances a biological sample may be attached to the shaft by a force stronger than the force applied between the biological sample and the cassette. In such an instance, when the cassette is moved away from the shaft, the biological sample may not detach properly and completely from the shaft.

In some such instances the biological sample may remain stuck to the shaft in a limited region wherein the force or forces attaching the sample to the shaft are stronger than the force or forces attaching the sample to the cassette. For example, the shaft may have a region that is less polished than other regions supporting the biological sample, and the less-polished region of the shaft may adhere to the biological sample stronger than the cassette. In another example, a core biopsy needle may have a sharp edge or a tiny sliver that may hook up to the sample at a point or in a limited region.

Thus, in some instances an attempt to detach a biological sample from a shaft by simply departing and moving away the cassette from the shaft in a direction vertical to the surface of the shaft on which the sample is supported, may end up with an undesired result of detaching the sample from the cassette. In some instances wherein the sample is attached strongly to the shaft at least in a point or in a limited region, the sample may be detached from the cassette in pealing-like progression, as the cassette is moved further and further away from the shaft.

It may therefore be advantageous to provide devices and methods for collecting onto a cassette a biological sample carried on a shaft or on a surface of a beam such as a scalpel or such as a biopsy needle having a mandrel and a notch. In some embodiments the cassette comprises an adhering surface configured to adhere to a biological sample when contacting the biological sample. According to an aspect of some embodiments, the method comprises
(a) providing a device having a base and a track, the track being configured to constrain a displacement of a movable member along a pre-defined trajectory relative to the base;
(b) installing onto the device a cassette and a beam carrying a biological sample, and
(c) displacing a movable member along the pre-defined trajectory, thereby displacing the biological sample in a direction parallel or tangential to the surface supporting the biological sample,
thereby detaching the biological sample from the surface of the beam and attaching the biological sample onto the cassette.

Displacing in parallel to the surface on which the biological sample is initially supported herein means displacing in a direction confined to a plane that is substantially parallel or tangential to the surface of the beam on which the biological sample is initially supported. In other words, a displacement that is parallel to the surface is substantially perpendicular to a displacement in a direction vertical to the surface.

Without being bound by a specific hypothesis, speculation or theory, it is envisaged that the force attaching the biological sample to the surface of the beam, e.g. a notch of a biopsy needle, may be characterized by high spatial variability. In other words, a sample may be attached to a supporting surface by a force which may be relatively strong in a point or over a small region. Such a strong force might overcome, in some instances, a competing force such as an adhering force of the sample to the adhering surface, so that an attempt to detach the sample from the surface of the beam would fail. However, when integrated over the entire surface of contact between the sample and the adhering surface of the cassette, the total force may be stronger than the integrated force attaching the biological sample to the shaft, and consequently may overcome, leading to proper detachment of the sample from the shaft, even in case of a local or a point attachment of the sample to the shaft which is relatively strong.

According to some embodiments the method further comprises, prior to the detaching step described above, slightly displacing the surface of the beam or the notch away from the adhering surface, in a direction substantially perpendicular to the adhering surface, by a distance that is smaller than about twice the thickness of the sample.

According to an aspect of some embodiments there is provided a device for collecting onto a cassette a biological sample carried on a surface of a beam. The device comprises a base, a gun house physically associated with the base and configured to house a handle of a beam configured to support a biological sample on a surface thereof. The device further comprises a cassette holder physically associated with the base and configured to house a cassette. The cassette comprises an adhering surface configured to adhere to a biological sample upon contacting or pressing onto the biological sample. The device further comprises a track, the track being configured to constrain a displacement of a movable member along a pre-defined trajectory relative to the base. The movable member is configured to move along the pre-defined trajectory, to effect a displacement of a biological sample supported on the surface of the beam tangentially to the surface, and to effect detachment of the biological sample from the beam and attachment of the biological sample onto the cassette.

This disclosure separately provides a method which can be used for detaching from a shaft a biological sample initially supported thereon and attaching the biological sample onto a cassette.

Further aspects of the present invention are exemplified in the following:
1. A method for collecting onto a cassette a biological sample carried on a surface of a beam, the method comprising:
   a. providing a device having a base and a track, the track being configured to constrain a displacement of a movable member along a pre-defined trajectory relative to the base;
   b. installing a cassette onto the device;
   c. installing a beam carrying a biological sample on a surface thereof onto the device;
   d. displacing a movable member along the pre-defined trajectory, thereby affecting a displacement of the biological sample in a direction parallel or tangential to the surface of the beam;
   thereby detaching the biological sample from the surface of the beam and attaching the biological sample onto the cassette.
2. The method of aspect 1 wherein the pre-defined trajectory is parallel or tangential to the surface of the beam.
3. The method of aspect 1 wherein the beam comprises a biopsy needle having a notch having a notch floor, the notch floor being configured for supporting a biological sample thereon.
4. The method of aspect 1 wherein the cassette comprises an adhering surface configured to adhere to a biological sample upon contacting or pressing onto the biological sample.
5. The method of aspect 4 wherein the pre-defined trajectory is parallel or tangential to the adhering surface.
6. The method of aspect 4 wherein the pre-defined trajectory is perpendicular to the adhering surface.
7. The method of aspect 1 wherein the movable member comprises the beam and the method comprises displacing the beam along the pre-defined trajectory relative to the base.
8. The method of aspect 7 wherein the cassette is stationary relative to the base during said step (d).
9. The method of aspect 8 wherein said step (d) comprises:
   i. positioning a wiper so that the biological tissue is supported by the surface of the beam between the wiper and the cassette, whereas the wiper is configured to prevent displacement of the biological tissue together with the beam when the beam is distanced from the cassette in a direction parallel to the surface of the beam, and
   ii. distancing the beam from the cassette along the pre-defined trajectory in a direction parallel to the surface of the beam, thereby effecting a displacement of the beam relative to the wiper and a consequent displacement of the biological tissue along the surface of the beam,
   thereby detaching the biological tissue from the surface of the beam.
10. The method of aspect 9 wherein the wiper is made of a rigid or semi-rigid material.
11. The method of aspect 9 wherein the material of the wiper is selected from the group consisting of polypropylene, polytetrafluoroethylene or Polyethylene terephthalate (PET).
12. The method of aspect 9 wherein the wiper is made of a soft and flexible material.
13. The method of aspect 12 wherein the material of the wiper is a sponge.
14. The method of aspect 7 wherein said step (d) comprises
   i. displacing the beam so that the biological sample supported on the beam contacts the cassette, thereby attaching the biological sample to the cassette, and
   ii. displacing the beam along the pre-defined trajectory so that the biological sample, being attached to the cassette, is dragged on the surface of the beam tangentially to the surface,
   thereby detaching the biological sample from the beam.
15. The method of aspect 14 wherein said step (i) comprises laterally displacing the beam in a lateral direction parallel to the adhering surface and revolving the beam around an axis perpendicular to the lateral direction.
16. The method of aspect 14 wherein the method further comprises, prior said step (ii), a step of distancing the beam from the cassette in a direction perpendicular to the surface of the beam.
17. The method of aspect 16 wherein said device comprises a cassette holder, the cassette holder being configured to be assembled with the cassette, and wherein said attaching of the biological sample to the cassette comprises
   i. moving the cassette holder from a first position wherein the cassette is distant from the needle bed to a second position wherein the cassette is proximal to the biological tissue and contacts the biological tissue supported in the notch, and
   ii. moving the cassette holder from the second position to a third position wherein the cassette is proximal said needle bed wherein the cassette contacts the biological tissue supported in the notch,
   thereby detaching the biological sample from the beam.
18. The method of aspect 1 wherein the movable member comprises the cassette.
19. The method of aspect 18 wherein the beam is stationary relative to the base.
20. The method of aspect 18 wherein said step (d) comprises:
   i. displacing the beam or the cassette so that the cassette contacts the biological sample supported on the beam, thereby attaching the biological sample to the cassette;
   ii. displacing the cassette along the pre-defined trajectory so that the biological sample, being attached to the cassette, is dragged on the surface of the beam tangentially to the surface,
   thereby detaching the biological sample from the beam.
21. The method of aspect 20 wherein said step (ii) comprises laterally displacing the cassette in a lateral direction parallel to the surface of the beam and revolving the cassette around an axis perpendicular to the lateral direction.
22. The method of aspect 20 wherein the method further comprises, prior to said step (ii), a step of distancing the cassette from the beam in a direction perpendicular to the surface of the beam.
23. The method of aspect 1 wherein the movable member does not comprise neither the beam nor the cassette.
24. The method of aspect 23 wherein during said step (d) the cassette and the beam are stationary relative to the base.
25. The method of aspect 23 wherein the movable member comprises a wiper configured to displace the biological tissue tangentially along the surface of the beam, and thereby to detach the biological tissue from the beam and attach the biological tissue to the cassette.
26. The method of aspect 25 wherein the wiper is made of a rigid or semi-rigid material and is shaped and dimensioned to slide tangentially along the surface of the beam thereby displacing the biological tissue.
27. A device for collecting onto a cassette a biological sample carried on a surface of a beam, said device comprising:
   a base;
   a gun house physically associated with said base and configured to house a handle of a beam configured to support a biological sample on a surface thereof;
   a cassette holder physically associated with said base and configured to house a cassette, said cassette comprising an adhering surface configured to adhere to a biological sample upon contacting or pressing onto the biological sample, and
   a track, said track being configured to constrain a displacement of a movable member along a pre-defined trajectory relative to said base,
      wherein said movable member is configured to move along said pre-defined trajectory, to effect a displacement of a biological sample supported on said surface of said beam tangentially to said surface, and to effect detachment of said biological sample from said beam and attachment of said biological sample onto said cassette.
28. The device of aspect 27 wherein said beam comprises a core biopsy needle having a notch having a notch floor, said notch floor being configured for supporting a biological sample thereon, and said surface comprises a portion of said notch floor.
29. The device of aspect 27 wherein said cassette is assembled with a sample sheet, said sample sheet comprising said adhering surface.
30 The device of aspect 27 wherein said pre-defined trajectory is parallel or tangential to said surface of said beam.
31. The device of aspect 27 wherein said pre-defined trajectory is parallel or tangential to said adhering surface.
32. The device of aspect 27 wherein said pre-defined trajectory is perpendicular to said adhering surface.
33. The device of aspect 27 wherein said movable member comprises said beam.
34. The device of aspect 33 further comprising a wiper positionable proximal to said pre-defined trajectory so that a biological tissue supported by said surface of said beam is between said wiper and said cassette, and said wiper is configured to prevent displacement of said biological tissue together with said beam when said beam is distanced from said cassette along said pre-defined trajectory in a direction parallel to said surface of said beam.
35. The device of aspect 34 wherein said wiper is associated with a cassette holder movable relative to said base between an open position and a closed position, said closed position being proximal to said pre-defined trajectory.
36. The device of aspect 34 wherein said beam comprises a biopsy needle having a notch and said pre-defined trajectory is perpendicular to a long axis of said biopsy needle.
37. The device of aspect 34 wherein said wiper is made of a rigid or semi-rigid material.
38. The device of aspect 37 wherein the material of said wiper is selected from the group consisting of polypropylene, polytetrafluoroethylene or Polyethylene terephthalate (PET).
39. The device of aspect 34 wherein the wiper is made of a soft and flexible material.
40. The device of aspect 39 wherein the material of the wiper is a sponge or made of soft silicone.
41. The device of aspect 34 wherein a distance between said wiper and said beam, when said beam is moved along said pre-defined trajectory, is smaller than about 1 mm.
42. The device of aspect 41 wherein a distance between said wiper and said beam, when said beam is moved along said pre-defined trajectory, is smaller than about 0.1 mm.
43. The device of aspect 40 wherein said wiper overlaps with said pre-defined trajectory, thereby frictionally sliding on said surface of said beam when said beam is displaced along said pre-defined trajectory.
44. The device of aspect 34 wherein said pre-defined trajectory is perpendicular to said adhering surface.
45. The device of aspect 33 wherein said gun house comprises said track.
46, The device of aspect 45 wherein said beam comprises a biopsy needle having a notch and said pre-defined trajectory is perpendicular to a long axis of said biopsy needle.
47. The device of aspect 46 wherein said gun house comprises a groove and a cradle, said groove defining said track and said cradle being configured to house a handle of said biopsy needle and being movable along said groove thereby displacing said biopsy needle along said pre-defined trajectory.
48. The device of aspect 47 wherein said groove is configured to rotate said cradle thereby rotating said biopsy needle along said long axis of said biopsy needle when said cradle is moved along said groove.
49. The device of aspect 45 wherein said beam comprises a biopsy needle having a notch and said pre-defined trajectory is parallel to a long axis of said biopsy needle.
50. The device of aspect 49 further comprising a needle bed having an alignment slot and configured to align said biopsy needle in said alignment slot when said biopsy needle is housed in said gun house, thereby confining a position of said biopsy needle at least in one direction, and wherein said cassette holder being configured to house said cassette, and being movable between well-defined positions relative to said needle bed, so that:
   in a first well-defined position said cassette and said needle bed are distant from one another;
   in a second well-defined position said cassette and said needle bed are proximal to one another so that said biopsy needle is supported on said needle bed between said needle bed and said cassette and contacts said cassette, and
   in a third well-defined position said cassette and said needle bed are proximal to one another and said biopsy needle is supported on said needle bed between said needle bed and said cassette and does not contact said cassette.
51. The device of aspect 50 further comprising a spring configured to support said cassette holder in said second and third well defined positions, said spring being energized in said second well-defined position, and being relaxed in said third well-defined position.
52. The device of aspect 27 wherein said movable member comprises said cassette.
53. The device of aspect 52 wherein said cassette holder comprises a groove, wherein said cassette, when assembled in said cassette holder, is movable and confined to displacements along said groove, said groove defining thereby said track.
54. The device of aspect 53 wherein said cassette is assembled with a sample sheet and said beam comprises a biopsy needle having a notch.
55. The device of aspect 53 wherein said pre-defined trajectory defined by said track is perpendicular to a long axis of said beam, when said beam is housed in said gun house.
56. The device of aspect 55 wherein said groove is configured to rotate said cassette around an axis parallel to said long axis of said biopsy needle when said cassette is moved along said groove.
57. The device of aspect 27 wherein said movable member does not comprise neither said beam nor said cassette.
58. The device of aspect 57 wherein said movable member comprises wiper, said wiper being associated with a cassette holder movable relative to said base between an open position and a closed position, said wiper being thereby configured to displace a biological tissue supported by said beam tangentially to said surface of said beam when said cassette holder is moved from said open position to said closed position.
59. The device of aspect 58 wherein said beam comprises a biopsy needle having a notch and said pre-defined trajectory is perpendicular to a long axis of said biopsy needle.
60. The device of aspect 58 wherein said wiper is made of a rigid or semi-rigid material.
61. The device of aspect 60 wherein the material of said wiper is selected from the group consisting of polypropylene, polytetrafluoroethylene or Polyethylene terephthalate (PET).
62. The device of aspect 58 wherein the wiper is made of a soft and flexible material.
63. The device of aspect 62 wherein the material of the wiper is a sponge or made of soft silicone.
64. The device of aspect 58 wherein a distance between said wiper and said beam, when said wiper is moved along said pre-defined trajectory, is smaller than about 1 mm.
65. The device of aspect 64 wherein a distance between said wiper and said beam, when said wiper is moved along said pre-defined trajectory, is smaller than about 0.1 mm.
66. The device of aspect 62 wherein said pre-defined trajectory of said wiper overlaps with said beam, said wiper thereby frictionally sliding on said surface of said beam when said wiper is displaced along said pre-defined trajectory.

Aspects of some embodiments are further described in the specification hereinbelow and in the appended claims.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

As used herein, the indefinite articles “a” and “an” mean “at least one” or “one or more” unless the context clearly dictates otherwise.

### BRIEF DESCRIPTION OF THE FIGURES

Some embodiments herein are described with reference to the accompanying figures. The description, together with the figures, makes apparent to a person having ordinary skill in the art how embodiments may be practiced. The figures are for the purpose of illustrative discussion and no attempt is made to show structural details of an embodiment in more detail than is necessary for a fundamental understanding of the teachings herein. For the sake of clarity, some objects depicted in the figures are not to scale.

In the Figures:

FIG. 1A (prior art) schematically depicts a core biopsy needle prior to obtaining a sample from an organ;

FIG. 1B (prior art) schematically depicts a core biopsy needle when the mandrel is advanced into the organ and organ tissue fills the notch;

FIG. 1C (prior art) schematically depicts a core biopsy needle when the cannula is advanced over the mandrel;

FIG. 1D (prior art) schematically depicts a core biopsy needle when the cannula is pulled back to expose a sample tissue on the notch;

FIG. 2A schematically depicts an embodiment of a device for collecting onto a cassette housed in a cassette holder a biological sample;

FIG. 2B schematically depicts the pedestal of the device of FIG. 2A, wherein the cassette holder is in the first position,

FIG. 2C schematically depicts the pedestal of the device of FIG. 2A, wherein the cassette holder is in the second and third positions,

FIG. 3 schematically depicts an embodiment of a device for collecting onto a cassette a biological sample, wherein the movable member is the biopsy needle and the pre-determine trajectory is perpendicular to the needle;

FIG. 4 schematically depicts an embodiment of a device for collecting onto a cassette a biological sample, wherein the movable member is the cassette;

FIG. 5A schematically depicts a mutual arrangement of the cassette and the biopsy needle of FIG. 4, as the cassette is displaced between positions along the track;

FIG. 5B schematically depicts another arrangement of the cassette and the biopsy needle of FIG. 4, as the cassette is displaced along the track;

FIG. 5C schematically depicts yet another arrangement of the cassette and the biopsy needle of FIG. 4, as the cassette is displaced along the track;

FIG. 5D schematically depicts yet another arrangement of the cassette and the biopsy needle of FIG. 4, as the cassette is displaced along the track;

FIG. 5E schematically depicts yet another arrangement of the cassette and the biopsy needle of FIG. 4, whereas the cassette departs from the biopsy needle;

FIG. 6A schematically depicts an embodiment of another cassette holder holding a cassette and rolling around the needle.

FIG. 6B schematically depicts the cassette holder of FIG. 6A in an open position.

FIG. 7A schematically depicts an embodiment of a device for collecting onto a cassette a biological sample, wherein the movable member may be a wiper associated with a movable lever;

FIG. 7B schematically depicts the device of FIG. 7A wherein the lever is in a closed position;

FIG. 8 schematically depicts the device of FIG. 7A wherein the lever is in an open position;

FIG. 9A schematically depicts the device of FIG. 7A wherein the lever is in the closed position and wherein the wiper is rigid or soft,

FIG. 9B schematically depicts an embodiment of a device different from the device of FIG. 7A by having a wiper that is soft,

FIGS. 10 to 15 schematically depict embodiments of yet further devices for collecting onto a cassette a biological sample, and

FIGS. 16, 17A and 17B schematically depict embodiments of a cleaning crib of the present invention.

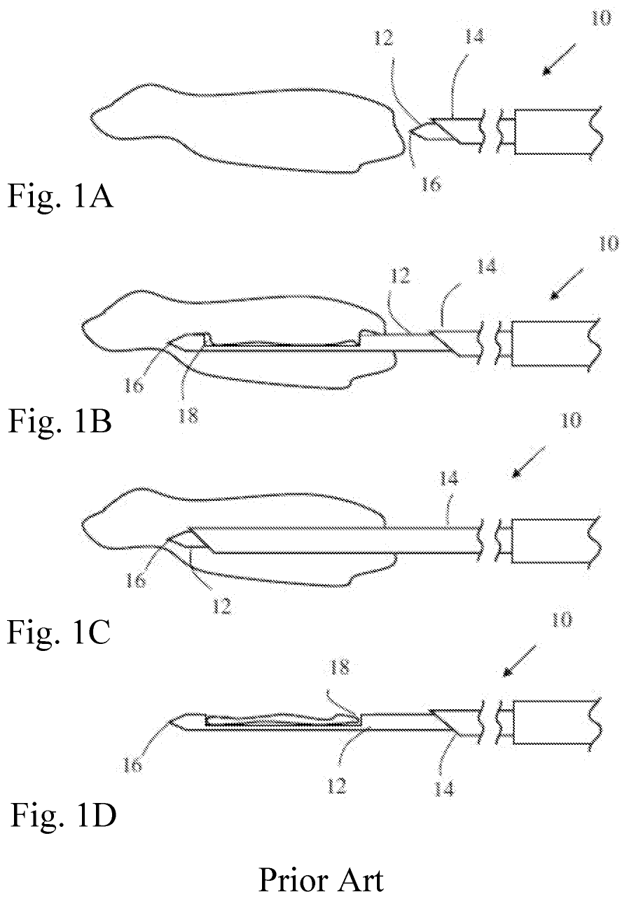

### DETAILED DESCRIPTION OF SOME EMBODIMENTS

The principles, uses and implementations of the teachings herein may be better understood with reference to the accompanying description and figures. Upon perusal of the description and figures present herein, one skilled in the art is able to implement the teachings herein without undue effort or experimentation.

Before explaining at least one embodiment in detail, it is to be understood that the disclosure is not necessarily limited in its application to the details of construction and the arrangement of the components and/or methods set forth herein. Other embodiments may be practiced, and an embodiment may be carried out in various ways. The phraseology and terminology employed herein are for descriptive purpose and should not be regarded as limiting.

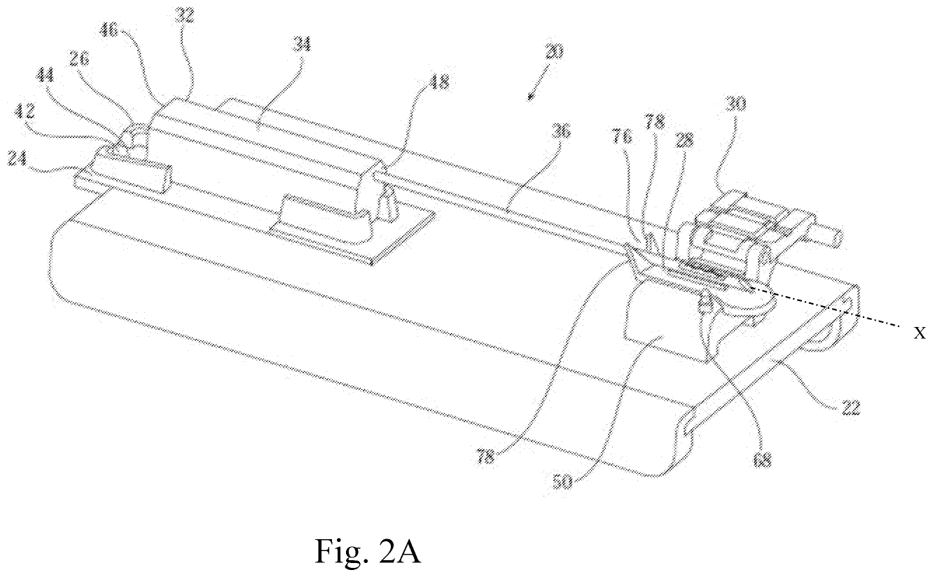

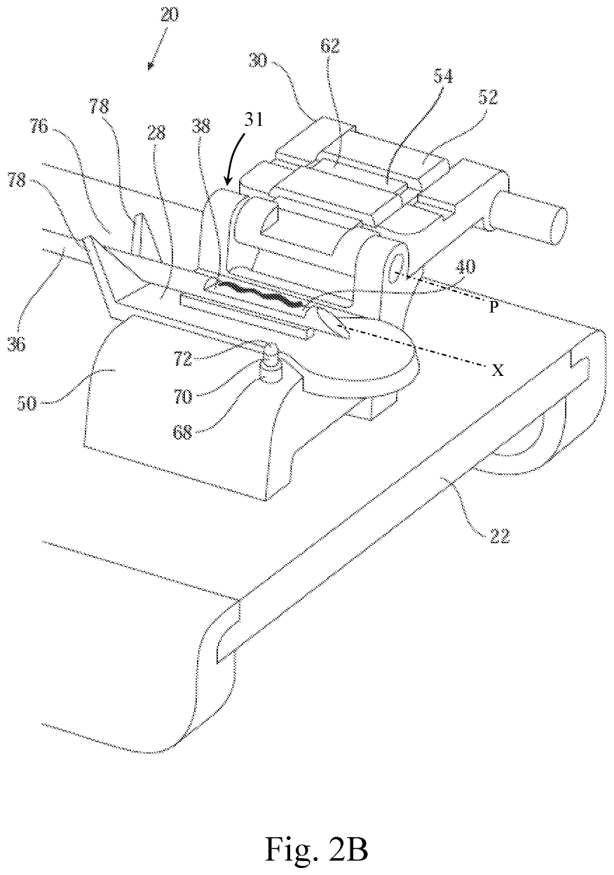

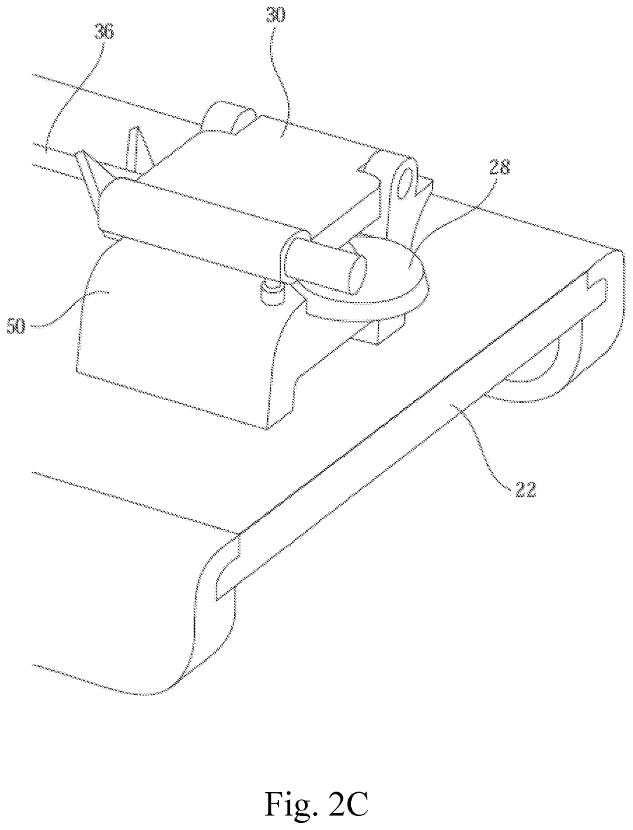

An embodiment of a device 20 for collecting onto a cassette a biological sample as described herein may be schematically depicted in FIGS. 2A-2C. Device 20 comprises a base 22, a gun house 24, a needle bed 28 and a cassette holder 30.

Gun house 24 comprises a gun frame 26 configured for receiving therein a beam suitable for carrying a biological sample on a surface thereof. In some embodiments gun house 24 may be configured to house a biopsy gun 32, having a gun handle 34 and a biopsy needle 36 suitable for carrying a biopsy sample. In some embodiments biopsy needle 36 (depicted schematically in more detail in FIG. 2B) may have a notch 38 having a surface, e.g. a notch floor 40, configured to house support and/or carry a biological sample thereon, as explained above.

Gun frame 26 may have a shape and internal dimensions that fit a shape and external dimensions of gun handle 34, so that when gun handle 34 is suitably placed inside gun frame 26, biopsy gun 32 can be secured in gun house 24. When biopsy gun 32 is secured in gun house 24, gun frame 26 in at least certain embodiments may be arranged to prevent substantial sideways movements of biopsy gun 32. Gun frame 26 may be suited to enable a backward and forward displacement of biopsy gun 32, substantially along a longitudinal axis X of biopsy needle 36 that passes through the biopsy needle. Such backward and forward displacement may be along a gap 42 of the gun house 24. Thus gun frame 26 defines a track 44 coinciding with gap 42, track 44 enabling displacements of biopsy gun 32 along a pre-defined trajectory (i.e. along gap 42) relative to base 22. When biopsy gun 32 is suitably placed and secured in gun house 24, biopsy needle 36 may be supported on needle bed 28. Biopsy gun 32 may be positioned in gun house 24 in a forward position, as is depicted in FIG. 2A, whereas gap 42 is substantially adjacent a back rear axial side 46 of gun handle 34. Alternatively or additionally, biopsy gun 32 may be placed in gun house 24 in a backward position (not shown) so that gap 42 is substantially adjacent a front axial side 48 of gun handle 34 (that is, the side of gun handle 34 that is closer to the distal tip). Biopsy gun 32 may also be displaced, e.g. by sliding, between a forward position and a backward position. Biopsy gun 32 may further be placed in any desired intermediate position between the forward position and the backward position.

Cassette holder 30 may be pivotally connected at a hinge member 31 to a pedestal 50, which in turn may be fixed to base 22. Pedestal 50 may be configured to support needle bed 28 at an elevated position relative to base 22, so that, when biopsy gun 32 is secured in gun house 24, biopsy needle 36 is positioned just above needle bed 28, to be supported thereon. Cassette holder 30 may be configured to be pivotally rotated about a pivot axis P of hinge member 31, in relative upward and downward directions relative to base 22. Such rotational movements of the lever may be defined as occurring between a first possibly well-defined open position, schematically depicted in FIGS. 2A and 2B, wherein cassette holder 30 is substantially distant from needle bed 28, and a second and a third possibly well-defined closed positions, schematically depicted together in FIG. 2C, wherein cassette holder 30 and needle bed 28 are situated substantially proximal to one another.

Cassette holder 30 may be configured to be assembled with a cassette 52 having an adhering surface 54 as is depicted in FIG. 2B. In some embodiments cassette 52 may be configured to be equipped with a sample sheet 62, having adhering surface 54. Exemplary, non-limiting embodiments of cassette holder 30 and embodiments of sample sheet 62 are described in detail in e.g. Application '031.

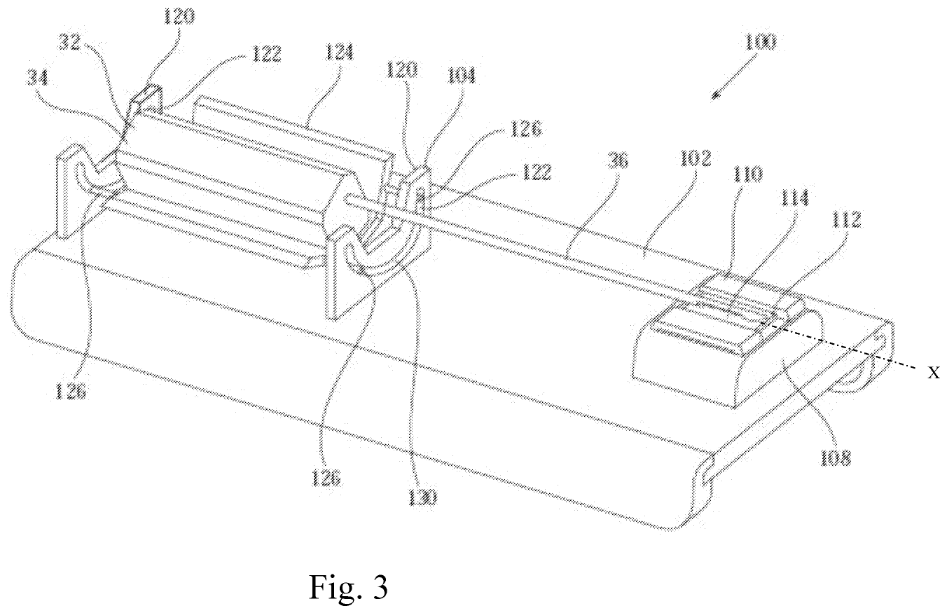

Pedestal 50 may include a stopper pillar 68 extending upwards from pedestal 50. Stopper pillar 68 may in turn include a spring plunger 70 such as e.g. GN 614.4 by Otto Ganter GmbH & Co. KG, aligned vertically on pedestal 50. Spring plunger 70 comprises a top ball 72 supported by an internal spring (not shown) that presses onto ball 72 upwards. When cassette holder 30 is moved from the first open position to one of the second or third closed positions as is depicted in FIG. 3C, cassette holder 30 may be supported by spring plunger 70. In embodiments where cassette holder 30 may be supported by a spring member such as spring plunger 70, cassette holder 30 may be pressed downwards against ball 72 so that the internal spring of spring plunger 70 may be substantially fully compressed, to the second position of cassette holder 30, thereby enabling contact between core biopsy needle 36 and adhering surface 54. If core biopsy needle 36 supports a biological sample on the notch, the biological sample may contact adhering surface 54, and thereby may be attached to adhering surface 54. In some embodiments the biological sample may even be pressed between the notch and adhering surface 54, thereby being attached to adhering surface 54.

When the pressing of cassette holder 30 against the spring member is released, in the example of spring plunger 70—the internal spring of spring plunger 70 may push upwards ball 72 and cassette holder 30 to the third position, in which a position of cassette holder 30 is slightly higher than a position of cassette holder 30 in the second position, so that the core biopsy needle 36 does not contact adhering surface 54. In some embodiments, adhering surface 54 may be lifted above core biopsy needle 36 in the third position (relative to the second position) by a distance between about 0.1 mm and about 3 mm. In some embodiments, adhering surface 54 may be lifted above core biopsy needle 36 in the third position relative to the second position by a distance between about 0.3 mm and about 1 mm. In some embodiments, adhering surface 54 may be lifted above core biopsy needle 36 by a distance that is relative to the diameter D of the core biopsy needle, e.g. between about 0.1 D and about 3 D. In some embodiments, adhering surface 54 may be lifted above core biopsy needle 36 by a distance between about 0.3 D and about D. In some embodiments, adhering surface 54 may be lifted above core biopsy needle 36 by a distance smaller than about 2 D. The distance by which adhering surface 54 is lifted above core biopsy needle 36 in the third position relative to the second position may be dictated by the compression distance of the spring member, the position of the spring member relative to the pivot of cassette holder 30 and the position of core biopsy needle 36, when supported by needle bed 28, relative to the pivot of cassette holder 30, as is clearly understood by a person skilled in the art from the arched type of displacement of cassette holder 30 around the hinge member.

In some embodiments, pedestal 50 comprises a source of a magnetic field, such as a fixed magnet or an electromagnet, located underneath needle bed 28 and configured to apply an attraction force on core biopsy needle 36 downwards towards needle bed 28. Such a magnetic force may assist in some embodiments to stabilize core biopsy needle 36 in place, and may assist in preventing undesired vertical displacements of core biopsy needle 36 during the detachment of a biological sample from the notch. An alignment slot 76 between alignment shoulders 78 of needle bed 28 may be suited to align biopsy needle 36 by preventing displacements of biopsy needle 36 perpendicularly to the needle, thereby confining the biopsy needle to displacements substantially along the longitudinal axis X of the needle. Some exemplary non-limiting embodiments of pedestal 50 having a fixed magnet as described above are described in detail in e.g. Application '031.

For use, and for adequate collection of a core biopsy sample from a core biopsy needle onto a cassette using device 20, the following steps may be employed in at least certain embodiments of the present disclosure.
(a) Cassette 52 may be equipped with sample sheet 62 and assembled in cassette holder 30, as is described above.
(b) Biopsy gun 32, carrying a sample tissue on or overlaying notch floor 40 of notch 38 of biopsy needle 36, may be placed in gun house 24 so that the notch of biopsy needle 36 faces substantially upwards. Biopsy needle 36 may be supported on needle bed 28, as is depicted in FIG. 2B. Biopsy gun 32 may be placed in gun house 24 in a forward position, so that gap 42 is adjacent the back side of gun handle 34, substantially as depicted in FIG. 2A. In some embodiments, a magnet may apply a magnetic force on biopsy needle 36, attracting biopsy needle 36 downwards towards needle bed 28 and assisting stabilizing biopsy needle 36 thereon.
(c) Cassette holder 30 may be lowered, e.g. by hand, from the open first position, and may be pressed downwards to the closed second position, as is schematically depicted in FIG. 2C. When sample sheet 62 is brought to contact with the sample tissue in the notch of biopsy needle 36, the sample tissue may adhere to sample sheet 62.
(d) Cassette holder 30 may then be released and pushed back to the closed third position by a spring member such as spring plunger 70. Adherence of the sample tissue to sample sheet 62 may be complete, meaning that the sample tissue is fully attached to the sample sheet and consequently that the sample tissue is completely detached from the notch. In some instances however adherence of the sample tissue to sample sheet 62 may not be complete meaning that the sample tissue is still attached to the notch, over an entire region or over a partial region of the sample sheet.
(e) When cassette holder 30 is in the third position and the sample sheet being slightly elevated above the core biopsy needle, biopsy gun 32 may be displaced horizontally, inside gun frame 26, from the forward position to the backward position, thereby tearing the sample tissue from the notch.
(f) When the sample tissue is thereby completely detached from the notch, cassette holder 30 may be lifted to the first position, allowing disassembly of cassette 52 from cassette holder 30, and disassembly of sample sheet 62 from cassette 52 and the taking of sample sheet 62 with the sample tissue thereon for further process.

In some embodiments, Biopsy gun 32 may be placed in gun house 24 in step (b) above, in a backward position, so that gap 42 is adjacent the front side 48 of gun handle 34. Consequently, in step (e) above, when cassette holder 30 is in the third position and the sample sheet being slightly elevated above the core biopsy needle, biopsy gun 32 may be displaced horizontally, along the longitudinal axis X of biopsy needle 36, inside gun frame 26, from the backward position to the forward position, thereby tearing the sample tissue from the notch. In some embodiments, Biopsy gun 32 may be placed in gun house 24 in step (b) above, in an intermediate position, namely between the forward position and the backward position, e.g. in the middle of gun frame 26. Consequently, in step (e) above, when cassette holder 30 is in the third position and the sample sheet being slightly elevated above the core biopsy needle, biopsy gun 32 may be displaced horizontally, along the longitudinal axis X of biopsy needle 36, inside gun frame 26, from the intermediate position backward and/or forward, to the backward position and/or to the forward position, (or vice versa), thereby tearing the sample tissue from the notch.

FIG. 3 schematically depicts an embodiment of a device 100 for collecting onto a cassette having an adhering surface, a biological sample carried on a shaft or on a surface of a beam or on a biopsy needle. In some embodiments the cassette may comprise a cassette including a sample sheet configured to adhere to a biological sample upon contacting the biological sample, as explained below.

Device 100 comprises a base 102 and a gun house 104 configured to support a biopsy gun 32, having a gun handle 34 and a biopsy needle 36 that extends along a longitudinal axis X. Device 100 further comprises a cassette house 108 configured to house a cassette 110. In some embodiments the cassette comprises a sample sheet 112, comprising e.g. a mesh film of cellulose esters. In some embodiments the cassette comprises an adhering surface 114 configured to adhere to a biological sample upon contacting the biological sample. A detailed description of some embodiments of cassette 110 is provided in Patent Application PCT/IL2013/050031, incorporated herein by reference. The cassette house 108 is fixed to the base 102.

Gun house 104 comprises vertical walls 120 fixed to the base 102, which comprise each a respective groove 122. Gun house 104 further comprises a cradle 124. Cradle 124 may be configured to house a gun handle 34 of biopsy gun 32, and with reference to axis X when housing a biopsy gun thereupon, may be defined as being disposed between the vertical walls 120, which are located on both its axial sides. The cradle may be designed to include pins 126 that are configured to slide inside the grooves 122, thereby allowing cradle 124 to swing and move along a track 130 defined by the grooves 122 relative to vertical walls 120, relative to base 102 and relative to cassette house 108. With reference to axis X when housing a biopsy gun upon the cradle, the track 130 may be formed in a plane generally perpendicular to axis X and may be formed about an axis coinciding or generally parallel to axis X. When a cassette 110 is suitably housed in cassette house 108 and a biopsy gun is suitably housed upon cradle 124 and the cradle is displaced along track 130 defined by grooves 122, biopsy needle 36 may be displaced between initial and terminal positions relative to cassette 110. Such initial and terminal positions may correspond to opposing ends of track 130.

Collecting a core biopsy sample initially carried by biopsy gun 32 onto cassette 110 may be accomplished as follows: cassette 110, comprising sample sheet 112 is suitably positioned in cassette house 108, whereas adhering surface 114 of sample sheet 112 faces upwards, substantially as depicted in FIG. 3. Cradle 124 is displaced along grooves 122, and positioned in one of the ends of track 130. Biopsy gun 32, carrying a biopsy sample in the notch of biopsy needle 36 (not shown in this Figure) is suitably housed in cradle 124, substantially as depicted in FIG. 3. When cradle 124 is positioned in one of the ends of track 130 as described above, core biopsy needle 36 is generally above cassette 110, whereas the notch faces generally downwards, towards cassette 110 and towards adhering surface 114. According to some embodiments there is substantially no contact between the biological sample in the notch and sample sheet 112. According to some embodiments the biological sample contacts the sample sheet when biopsy gun 32, carrying the biopsy sample in the notch, is suitably housed in cradle 124.

Cradle 124 may then be smoothly displaced, e.g. by hand, along track 130, namely along grooves 122, from the one end thereof to the other end thereof. In some embodiments the cradle may be displaced by a mechanical transmission system such as an electric motor and a suitable gear, and/or according to any technique known in the art. When the cradle is displaced along track 130, biopsy needle 36 and the notch thereof, are displaced sidewise while being displaced downwards and then upwards. Grooves 122 are configured to align biopsy needle 36 to contact adhering surface 114 at least when the cradle is in a median portion of the track, namely between the one end and the other end of track 130. While being displaced as described above, biopsy needle 36 also rotates around the longitudinal axis X of the needle, (perpendicularly to the pre-determined trajectory defined by track 130). Thus, a smearing effect is obtained when cradle 124 is displaced along track 130, as the notch nears the sample sheet, then contacts the sample sheet while being further displaced sidewise and while being slightly rotated along the needle axis, and then as the notch departs from the sample sheet while the biological sample remains stuck to the sample sheet. The rotation of biopsy needle 36 and the notch thereof while smearing the biological sample on the sample sheet facilitates and enhances the adherence of the biological sample to the sample sheet. In the embodiment of device 100 thus relative large portions of the circumference of the needle cross-section—and possibly relative larger portions of the biological sample on the notch—may come in contact with the sample sheet. Also, the sidewise displacement of the notch relative to the sample sheet may facilitate dragging of the sample sidewise from the notch, in a direction generally parallel to the notch floor, thus facilitating removing the sample from the notch. Employing lateral displacement between the biopsy needle and the sample sheet during collecting the sample from the needle onto the sample sheet may therefore be advantageous in some embodiments, compared to methods that do not employ such sidewise displacement, such as some methods disclosed in Patent Application PCT/IL2013/050031.

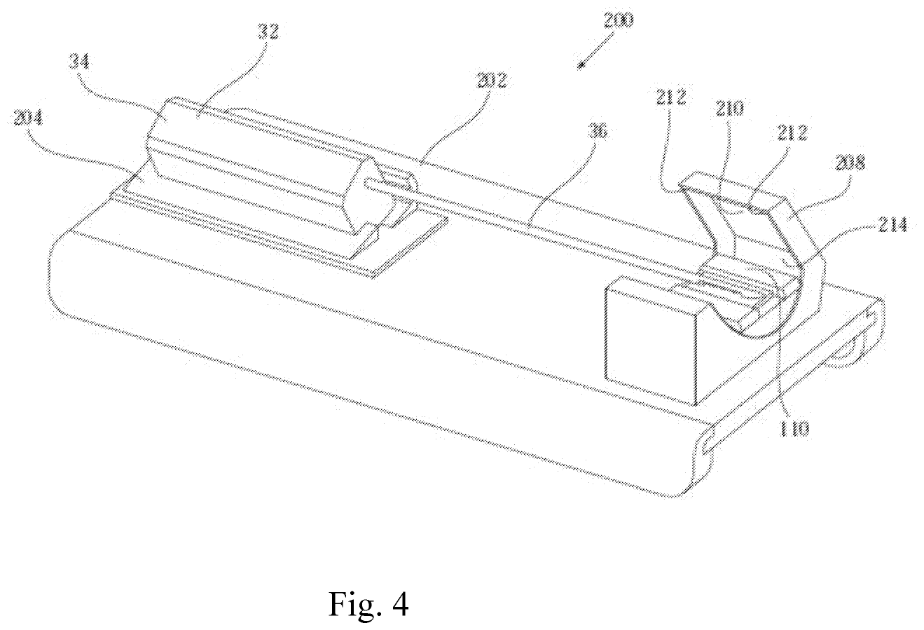

An embodiment of a device 200 for collecting onto a cassette a biological sample carried on a shaft, is schematically depicted in FIG. 4. Device 200 is configured to allow collecting of a sample onto a cassette using lateral displacement and a smearing effect as described above in FIG. 3. In device 200 the smearing effect may be achieved by displacing the cassette, while the biopsy gun remains stationary.

Device 200 comprises a base 202 and a gun house 204, fixed to the base and configured to support biopsy gun 32. Device 200 further comprises a cassette holder 208 configured to house a cassette 110. Cassette holder 208 may be fixed to base 202. Cassette holder 208 comprises a groove 210 descended between two rims 212. Groove 210 has a width that fits in dimensions to a width of cassette 110, thereby the groove is configured to confine the cassette, when suitably housed in the groove, to a pre-determined track 214 defined by the groove. Displacing the cassette along track 214, namely along groove 210 of cassette holder 208 substantially displaces cassette 110 with sample sheet 112 therein between positions relative to core biopsy needle 36 and the notch thereof.

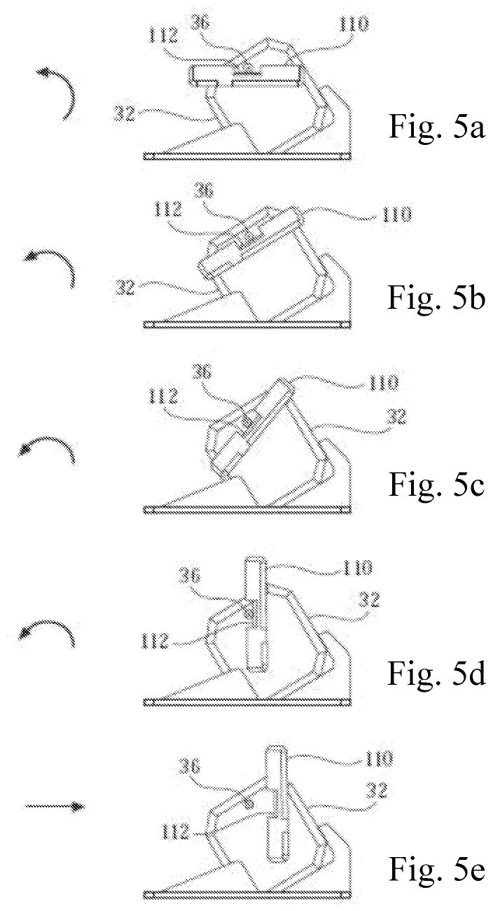

FIGS. 5A-5E schematically depict mutual arrangements of cassette 110 and biopsy gun 32 and biopsy needle 36 as the cassette is displaced between positions along track 214. In FIGS. 5A, 5B, 5C and 5D, respectively, the cassette substantially rotates around the biopsy needle while the notch and surrounding portions of the needle along the circumference of the needle are in contact with the sample sheet in the cassette. Cassette 100 rotates around biopsy needle 36 so that biopsy needle and the notch thereof contact sample sheet 112 along a substantially one line. In other words, biopsy needle 36 does not roll along sample sheet 112 but rather rotates while sliding at a single location of sample sheet 112. Consequently, a lateral displacement is effectively achieved between the circumference of biopsy needle 36 and sample sheet 112. Such lateral displacement assists in dragging a biological sample supported on the notch sidewise, in a direction parallel to the notch floor, thus facilitating removal of the sample from the notch onto the sample sheet 112.

In the position depicted in FIG. 5E cassette 110 departs from biopsy needle 36. Consequently, a biopsy sample initially supported on the notch may effectively be smeared on sample sheet 112, thereby enhancing adherence of the biological sample to the cassette.

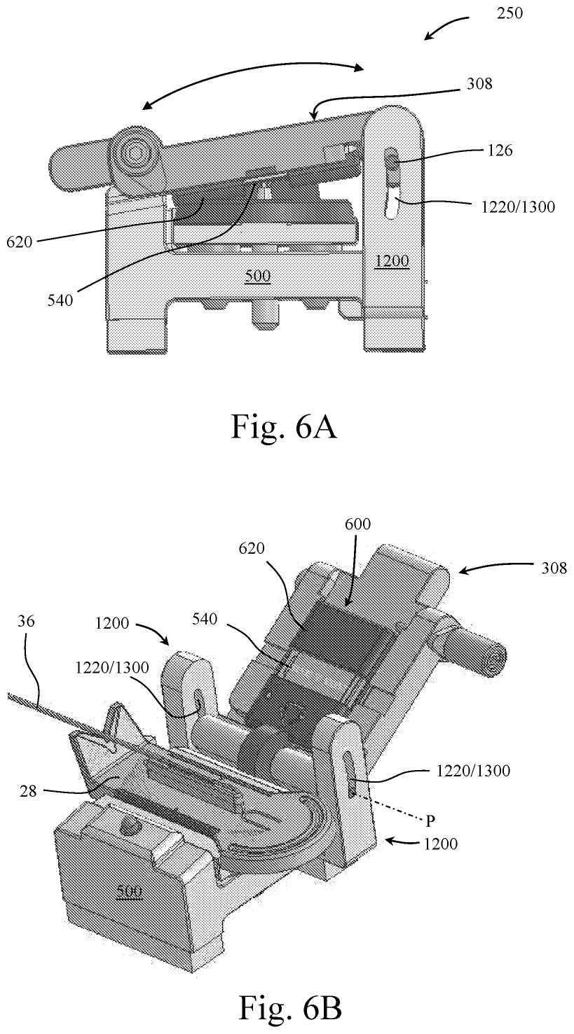

FIG. 6A schematically depicts an embodiment of a device 250 for collecting a biological sample initially carried on a surface of a beam—e.g. on a notch floor (not shown in this Figure) of biopsy needle 36—onto a cassette comprising an adhering surface. Biopsy needle 36 may be supported on a needle bed 280, which in turn may be supported by a pedestal 500 of the device. The device has a cassette holder 308, which includes a cassette 600 that is equipped with a sample sheet 620 that has an adhering surface 540 for collecting a sample. The collecting of the sample in this embodiment may be performed by displacing the cassette 600, while the biopsy gun remains static. In device 250, a rolling movement of the cassette holder 308 relative to the biopsy needle 36 may be performed. The rolling movement may be accomplished as seen in this example by providing two side walls 1200 that project up from the pedestal 500. Each side wall may be formed with a groove 1220 forming an arched track 1300 and the cassette holder 308 may comprise a hinge member 31 having a pivot axis P and pins 126 coaxial with axis P and at opposing axial sides of the hinge member, which are configured each to slide inside a respective one of the grooves 1220 along the arched tracks. Thus, the rolling of the cassette holder 308 relative to biopsy needle 36 may be accomplished by combined movements of rotation of the cassette holder 308 about pivot axis P and sliding of the cassette holder 308 up or down along the arched tracks through engagement of the pins within their respective grooves 1220. FIG. 6B schematically depicts the cassette holder of FIG. 6A in an open position.

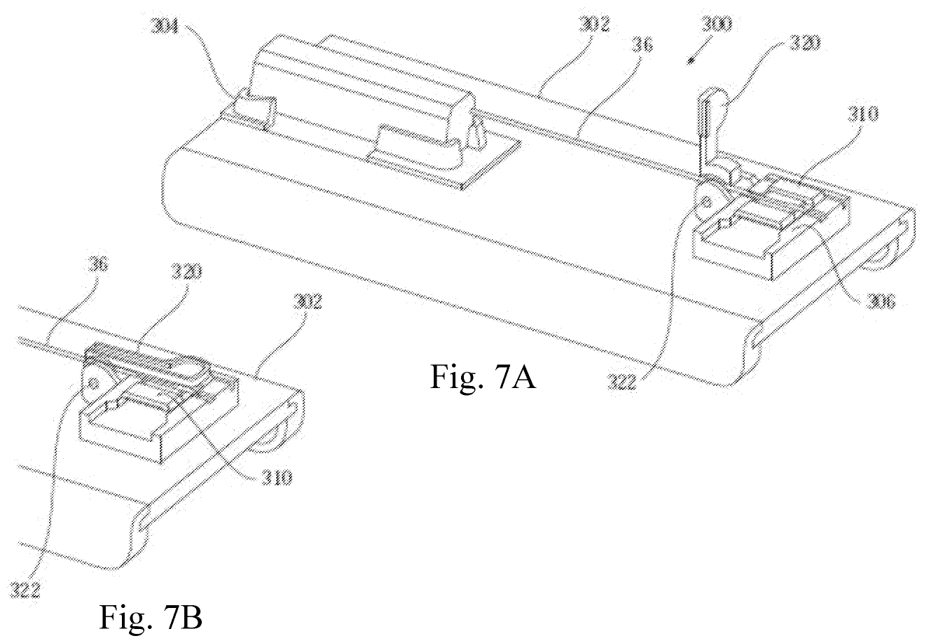

FIGS. 7A and 7B schematically depict an embodiment of a device 300 for collecting a biological sample initially carried on a surface of a beam—e.g. on notch floor 40 (not shown in this Figure) of biopsy needle 36—onto a cassette comprising an adhering surface such as a cassette assembled with a sample sheet. Device 300 comprises a base 302, a gun house 304 and a cassette holder 306. Gun house 304 is fixed to base 302 and configured to house a handle 34 of a biopsy gun 32 that may be generally stationary relative to base 302. Cassette holder 306 is fixed to base 302 and configured to house a cassette 310 therein. Device 300 further comprises a lever 320 pivotally attached to cassette holder 306. Lever 320 is configured to displace between positions along a trajectory determined by a pivot 322 used as an axis of rotation for lever 320. FIG. 7A schematically depicts device 300 wherein lever 320 is in an open position. FIG. 7B schematically depicts device 300 wherein lever 320 is in a closed position. Pivot 322 thus defines, in some embodiments, an arched track for lever 320 between the open position and the closed position.

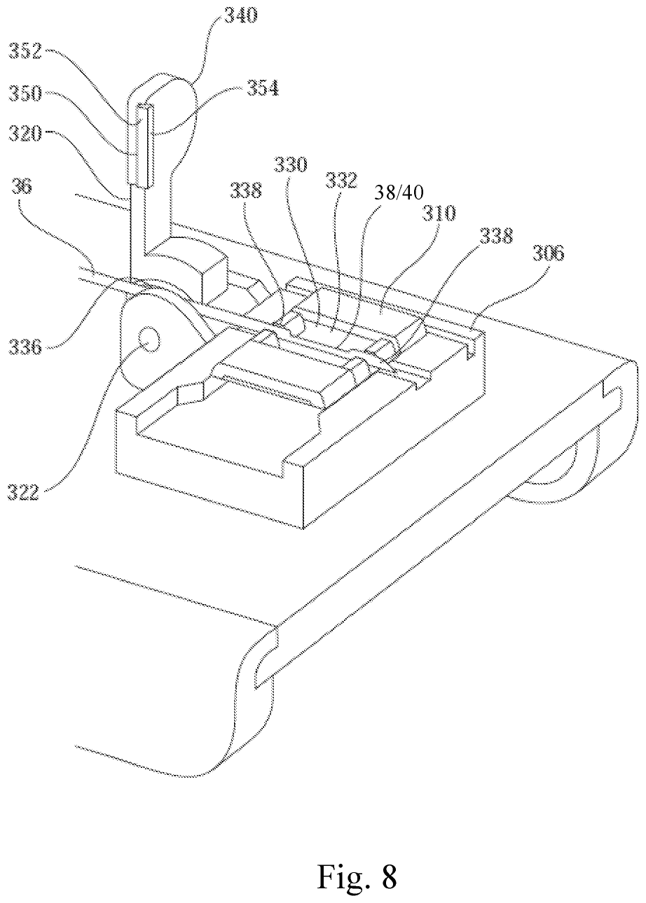

FIG. 8 schematically depicts cassette holder 306 and lever 320 in detail, in an open position. Cassette 310 is installed in cassette holder 306 so that a sample sheet 330 assembled in the cassette has an adhering surface 332 facing upwards. Biopsy needle 36 is installed onto device 300 so that notch 38 is above sample sheet 330, facing sidewise, that is to say that notch floor 40 is vertical and perpendicular to sample sheet 330. Cassette holder 306 comprises an alignment slit 336 positioned proximal to pivot 322 and fitting in width to a diameter of biopsy needle 36 and configured to align and stabilize biopsy needle 32 therein when biopsy needle 32 is suitably installed in device 300. Further, cassette 310 comprises two pairs of alignment grooves 338, fitting in width to the diameter of biopsy needle 36 and configured to align and stabilize notch 38 relative to cassette 310. In everyday practice biopsy needles might bend or otherwise physically distort during use, thus diverging from a straight line. Alignment slit 336 and alignment grooves 338 assist in restricting biopsy needle 36 to a substantially straight line suitably positioned in place above cassette 310.

Lever 320 comprises a handle 340 for gripping and displacing lever 320 by hand. Lever 320 further comprises a wiper 350 having a shape of a cuboid and having a face 352 being shorter than a length of notch 38, and arranged parallel to notch floor 40. Lever 320 is aligned relative to alignment slit 336 and to alignment grooves 338 so that when lever 320 is lowered to a closed position, wiper 350 is displaced tangentially to notch floor 40.

For use, and for detaching a biological tissue supported by notch floor 40 from the notch, and collecting the biological tissue on cassette 310, lever 320 may be lowered from an open position to a closed position whereas cassette 310 and needle 36, carrying a biological tissue, are adequately assembled in device 300. As lever 320 is lowered from an open position to a closed position, wiper 350 displaces the biological tissue supported by notch 38 along notch floor 40, parallel to notch floor 40, downwards towards sample sheet 330 of cassette 310. In some embodiments wiper 350 is made of a material inert to biological tissues so that wiper 350 does not chemically interact with the biological tissue supported on the notch and does not stick to the biological tissue as the wiper pushes the tissue towards the cassette. Consequently, when lever 320 is in the closed position, the biological tissue has been displaced on the notch floor and has contacted sample sheet 330 and has been adhered to sample sheet 330. Further, when lever 320 is moved back to the open position the biological tissue remains stuck to the sample sheet on the cassette.

According to some embodiments, detachment of the biological tissue from the notch may be obtained by moving lever 320 from the open position to the closed position and the corresponding displacement of wiper 350. Wiper 350 displaces and drags the biological tissue towards sample sheet 330 beyond the border of notch floor 40, thereby detaching the biological tissue from the notch. According to some embodiments, detachment of the biological tissue from the notch may be achieved by removing biopsy needle 36 from alignment slit 336 upwards, whereas lever 320 is in the closed position. In other words, the initial displacement of lever 320 from the open position to the closed position and the corresponding displacement of wiper 350 attaches the biological tissue to the sample sheet but does not necessarily detaches the biological tissue from the notch, whereas the biological tissue is disposed between wiper 350 and sample sheet 330. Alignment slit 336 and possibly alignment grooves 338 create a track of a defined trajectory (upwards) for needle 36, so as to affect a displacement of the biological tissue tangentially to the notch floor. Hence, when lever 320 is still in the closed position, biopsy needle 36 may be moved upwards in a direction parallel to the notch floor (and perpendicular to the adhering surface 332) thereby affecting dragging the biological tissue along the notch floor until detachment of the biological tissue from the notch is attained. The biological tissue is stuck to the sample sheet and remains stuck thereon as lever 320 may be displaced back from the closed position to the open position

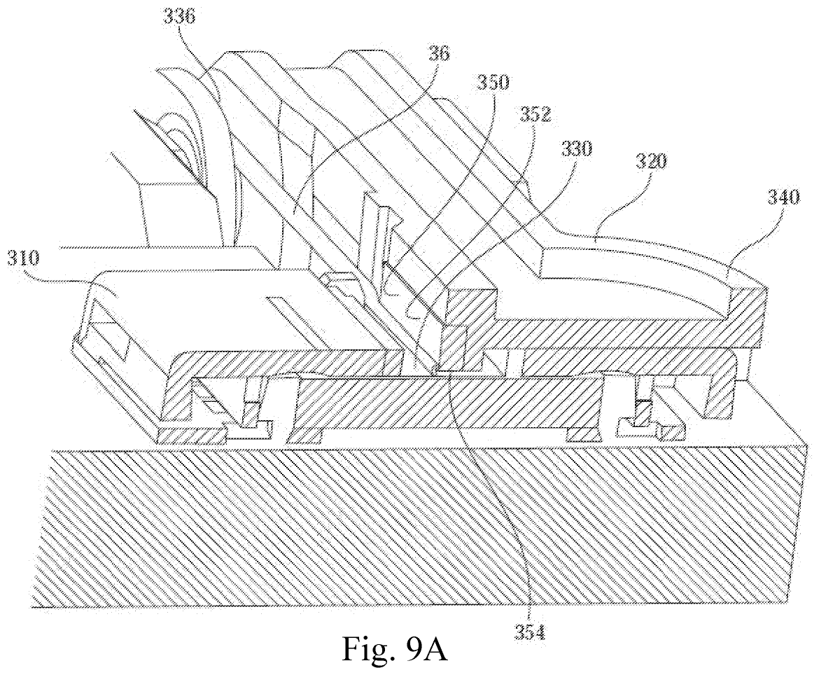

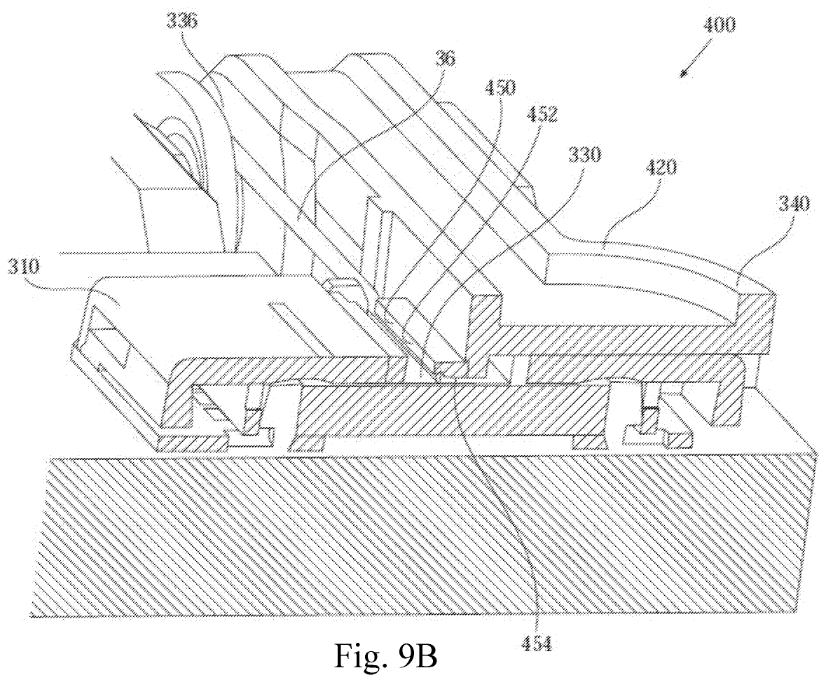

FIG. 9A schematically depicts a cross-section of device 300 having cassette 310 and biopsy needle 36 assembled therewith, and lever 320 being in a closed position. Face 352 of wiper 350 tangentially associate with notch floor 40, whereas a bottom face 354 of wiper 350 is proximal to sample sheet 330, above sample sheet 330, thereby ensuring contact between the biological tissue and the sample sheet and possibly even pressing the sample tissue onto the sample sheet. In some embodiments wiper 350 is made of rigid or semi-rigid materials such as polypropylene, polytetrafluoroethylene (Teflon®) or Polyethylene terephthalate (PET), for example Mylar®. In some embodiments wiper 350 is made of soft and flexible materials such as sponge or soft silicone.

FIG. 9B schematically depicts a cross-section of a device 400 which is different from device 300 in having a lever 420 having a soft wiper 450. Soft wiper 450 is further flexible and is shaped and dimensioned to cover biopsy needle 36 in a region of notch 38 when lever 320 is in the closed position, that is to say that a side face 452 of soft wiper 450 does not coincide and does not tangentially associate with notch floor 40. Further, a bottom face 454 of soft wiper 450 flexibly yields and squeezes above biopsy needle 36 as lever 420 is pressed downwards to the closed position. Thus, a portion of soft wiper 450 crumple above notch 38, thereby penetrating to notch 38 and displacing the biological tissue towards sample sheet 330. According to some embodiments biopsy needle may be displaced upwards so as to be removed from alignment slit 336 when lever 420 is in the closed position, thereby flexibly folding soft wiper 450 upwards. As biopsy needle 36 is so removed upwards, a portion of soft wiper 450 slides along notch floor 40, thereby displacing the biological tissue along notch floor 40 and detaching the biological tissue from notch floor 40.

Thus according to an aspect of some embodiments there is provided a device (20, 100, 200, 250, 300, 400) for collecting onto a cassette a biological sample carried on a surface of a beam. The device comprises a base (22, 102, 202, 302), a gun house (24, 104, 204, 304) physically associated with the base and configured to house a handle (34) of a beam configured to support a biological sample on a surface thereof. The device further comprises a cassette holder (30, 108, 208, 306) physically associated with the base and configured to house a cassette (52, 110, 310, 308). The cassette comprises an adhering surface (54, 114, 332, 540) configured to adhere to a biological sample upon contacting or pressing onto the biological sample. The device further comprises a track (44, 130, 214, 322, 336, 1300), the track being configured to constrain a displacement of a movable member along a pre-defined trajectory relative to the base. The movable member is configured to move along the pre-defined trajectory, to effect a displacement of a biological sample supported on the surface of the beam tangentially to the surface, and to effect detachment of the biological sample from the beam and attachment of the biological sample onto the cassette.

In some embodiments the beam comprises a core biopsy needle (36) having a notch (38) having a notch floor (40), the notch floor being configured for supporting a biological sample thereon, and the surface comprises a portion of the notch floor.

In some embodiments the cassette may be assembled with a sample sheet (62, 112, 330, 620), the sample sheet comprising the adhering surface.

In some embodiments (devices 20, 100, 200, 250, 300, 400) the pre-defined trajectory is parallel or tangential to the surface of the beam. In some embodiments (devices 20, 100, 200) the pre-defined trajectory is parallel or tangential to the adhering surface. In some embodiments (devices 300, 400) the pre-defined trajectory is perpendicular to the adhering surface. In some embodiments (devices 20, 100, 300, 400) the movable member comprises the beam.

In some embodiments, the device (300, 400) further comprises a wiper (350, 450) positionable proximal to the pre-defined trajectory (defined by alignment slit 336) so that a biological tissue supported by the surface of the beam is between the wiper and the cassette, and the wiper is configured to prevent displacement of the biological tissue together with the beam when the beam is from the cassette distanced (upwards along alignment slit 336) along the pre-defined trajectory in a direction parallel to the surface of the beam (notch floor 40). In some embodiments the wiper is associated with a lever (320) movable relative to the base between an open position and a closed position, the closed position being proximal to the pre-defined trajectory.

In some embodiments the beam comprises a biopsy needle having a notch and the pre-defined trajectory is perpendicular to a long axis of the biopsy needle.

In some embodiments (device 300) the wiper is made of a rigid or semi-rigid material. In some embodiments the material of the wiper is selected from the group consisting of polypropylene, polytetrafluoroethylene or Polyethylene terephthalate (PET). In some embodiments (devices 300, 400) the wiper is made of a soft and flexible material. In some embodiments, the material of the wiper is a sponge or made of soft silicone.

In some embodiments (device 300) a distance between the wiper and the beam, when the beam is moved along the pre-defined trajectory, is smaller than about 1 mm. In some embodiments (device 300) the distance between the wiper and the beam, when the beam is moved along the pre-defined trajectory, is smaller than about 0.1 mm.

In some embodiments (device 400) the wiper overlaps with the pre-defined trajectory, thereby frictionally sliding on the surface of the beam when the beam is displaced along the pre-defined trajectory. In some embodiments (devices 300, 400) the pre-defined trajectory is perpendicular to the adhering surface.

In some embodiments (devices 20, 100) the gun house (24, 104) comprises the track (44, 130)

In some embodiments (device 100) the beam comprises a biopsy needle having a notch and the pre-defined trajectory (130) is perpendicular to a long axis of the biopsy needle. In some embodiments the gun house comprises a groove (122) and a cradle (124), the groove defining the track (130) and the cradle being configured to house a handle (34) of the biopsy needle and being movable along the groove thereby displacing the biopsy needle along the pre-defined trajectory. In some embodiments (device 100) the groove is configured to rotate the cradle thereby rotating the biopsy needle along the long axis of the biopsy needle when the cradle is moved along the groove.

In some embodiments (device 20) the beam comprises a biopsy needle having a notch and the pre-defined trajectory is parallel to a long axis of the biopsy needle. In some embodiments the device (20) further comprises a needle bed (28) having an alignment slot (76) and configured to align the biopsy needle in the alignment slot when the biopsy needle is housed in the gun house, thereby confining a position of the biopsy needle at least in one direction. In some embodiments (device 20) the cassette holder comprises a lever (30) configured to house the cassette, the lever being movable between well-defined positions relative to the needle bed. In a first well-defined position (FIG. 2B) the cassette and the needle bed are distant from one another. In a second well-defined position (FIG. 2C) the cassette and the needle bed are proximal to one another so that the biopsy needle is supported on the needle bed between the needle bed and the cassette and contacts the cassette. In a third well-defined position (FIG. 2C) the cassette and the needle bed are proximal to one another and the biopsy needle is supported on the needle bed between the needle bed and the cassette and does not contact the cassette.

In some embodiments the device (20) further comprises a stopper (70) comprising a spring, the stopper being configured to support the lever (30) in the second and third well defined positions. The spring is energized (compressed or stretched) in the second well-defined position, and relaxed in the third well-defined position.

In some embodiments (device 200) the movable member comprises the cassette (110). In some embodiments the cassette holder (208) comprises a groove (212), wherein the cassette, when assembled in the cassette holder, is movable and confined to displacements along the groove, the groove defining thereby the track (214). In some embodiments the cassette may be assembled with a sample sheet (112) and the beam comprises a biopsy needle (36) having a notch (38). In some embodiments (device 200) the pre-defined trajectory defined by the track (214) is perpendicular to a long axis of the beam, when the beam is housed in the gun house. In some embodiments (device 200) the groove is configured to rotate the cassette around an axis parallel to the long axis of the biopsy needle when the cassette is moved along the groove.

In some embodiments (devices 300, 400) the movable member (350, 450) does not comprise neither the beam nor the cassette. In some embodiments (devices 300, 400) the movable member comprises a wiper (350, 450). The wiper is associated with a lever (320) movable relative to the base between an open position and a closed position. The wiper is thereby configured to displace a biological tissue supported by the beam (36) tangentially to the surface of the beam (notch floor 40) when the lever is moved from the open position to the closed position. In some embodiments (devices 300, 400) the beam comprises a biopsy needle having a notch and the pre-defined trajectory is perpendicular to a long axis of the biopsy needle.

In some embodiments (device 300) the wiper is made of a rigid or semi-rigid material. In some embodiments (device 400) the wiper is made of a soft and flexible material. In some embodiments (device 300) a distance between the wiper and the beam, when the wiper is moved along the pre-defined trajectory, is smaller than about 1 mm. In some embodiments (device 300) the distance between the wiper and the beam, when the wiper is moved along the pre-defined trajectory, is smaller than about 0.1 mm. In some embodiments (device 400) the pre-defined trajectory of the wiper (450) overlaps with the beam, the wiper thereby frictionally sliding on the surface of the beam when the wiper is displaced along the pre-defined trajectory.

According to an aspect of some embodiments there is provided a method for collecting onto a cassette a biological sample carried on a surface of a beam. The method comprises (a) providing a device (20, 100, 200, 300, 400) having a base (22, 102, 202, 302) and a track (44, 130, 214, 322, 336), the track being configured to constrain a displacement of a movable member (beam 36, cassette 110, wiper 350, wiper 450) along a pre-defined trajectory relative to the base. The method further comprises (b) installing a cassette (52, 110, 310) onto the device. The method further comprises (c) installing a beam (36) carrying a biological sample on a surface (40) thereof onto the device. The method further comprises (d) displacing the movable member along the pre-defined trajectory, thereby affecting a displacement of the biological sample in a direction parallel or tangential to the surface of the beam, thereby detaching the biological sample from the surface of the beam and attaching the biological sample onto the cassette.

In some embodiments (devices 20, 100, 200, 300, 400) the pre-defined trajectory is parallel or tangential to the surface of the beam.

In some embodiments the beam comprises a biopsy needle having a notch having a notch floor, the notch floor being configured for supporting a biological sample thereon. In some embodiments the cassette comprises an adhering surface configured to adhere to a biological sample upon contacting or pressing onto the biological sample.

In some embodiments (devices 20, 100, 200) the pre-defined trajectory is parallel or tangential to the adhering surface. In some embodiments (devices 300, 400) the pre-defined trajectory is perpendicular to the adhering surface.

In some embodiments (devices 20, 100, 300, 400) the movable member comprises the beam and the method comprises displacing the beam along the pre-defined trajectory relative to the base. In some embodiments (devices 100, 300, 400) the cassette is stationary relative to the base during the step (d).

In some embodiments (devices 300, 400) the step (d) comprises (i) positioning a wiper (350, 450) so that the biological tissue is supported by the surface of the beam between the wiper and the cassette. The wiper is configured to prevent displacement of the biological tissue together with the beam when the beam is distanced (along alignment slit 336) from the cassette in a direction parallel to the surface of the beam. Step (d) further comprises (ii) distancing the beam from the cassette along the pre-defined trajectory (along alignment slit 336) in a direction parallel to the surface of the beam, thereby effecting a displacement of the beam relative to the wiper and a consequent displacement of the biological tissue along the surface of the beam. The biological tissue is thereby detaced from the surface of the beam.

In some embodiments (device 300) the wiper is made of a rigid or semi-rigid material. In some embodiments (device 400) the wiper is made of a soft and flexible material.

In some embodiments (devices 20, 100) step (d) comprises a step (i) of displacing the beam so that the biological sample supported on the beam contacts the cassette, thereby attaching the biological sample to the cassette. Step (d) may further comprise a step (ii) of displacing the beam along the pre-defined trajectory so that the biological sample, being attached to the cassette, is dragged on the surface of the beam tangentially to the surface, thereby detaching the biological sample from the beam.

In some embodiments (device 100) the step (i) comprises laterally displacing the beam in a lateral direction parallel to the adhering surface and revolving the beam around an axis perpendicular to the lateral direction.

In some embodiments (device 20) the method further comprises, prior the step (ii), a step of distancing the beam from the cassette in a direction perpendicular to the surface of the beam. In some embodiments the device comprises a lever (30), the lever being configured to be assembled with the cassette. The attaching of the biological sample to the cassette comprises a step of moving the lever from a first position wherein the cassette is distant from the needle bed to a second position wherein the cassette is proximal to the biological tissue and contacts the biological tissue supported in the notch. The attaching of the biological sample to the cassette further comprises a step of moving the lever from the second position to a third position, wherein the cassette is proximal the needle bed wherein the cassette does not contact the beam.

In some embodiments (device 200) the movable member comprises the cassette. In some embodiments (device 200) the beam is stationary relative to the base. In some embodiments (device 200) the step (d) comprises: (i) displacing the beam or the cassette so that the cassette contacts the biological sample supported on the beam, thereby attaching the biological sample to the cassette. Step (d) further comprises: (ii) displacing the cassette along the pre-defined trajectory (track 214) so that the biological sample, being attached to the cassette, is dragged on the surface of the beam tangentially to the surface, thereby detaching the biological sample from the beam.

In some embodiments (device 200) the step (ii) comprises laterally displacing the cassette in a lateral direction parallel to the surface of the beam and revolving the cassette around an axis perpendicular to the lateral direction. In some embodiments the method further comprises, prior to the step (ii), a step of distancing the cassette from the beam in a direction perpendicular to the surface of the beam.

In some embodiments (devices 300, 400) the movable member (wiper 350, wiper 450) does not comprise neither the beam nor the cassette. In some embodiments (device 300), during the step (d) the cassette and the beam are stationary relative to the base. In some embodiments the movable member comprises a wiper configured to displace the biological tissue tangentially along the surface of the beam, and thereby to detach the biological tissue from the beam and attach the biological tissue to the cassette. In some embodiments the wiper (350) is made of a rigid or semi-rigid material and is shaped and dimensioned to slide tangentially along the surface of the beam thereby displacing the biological tissue.

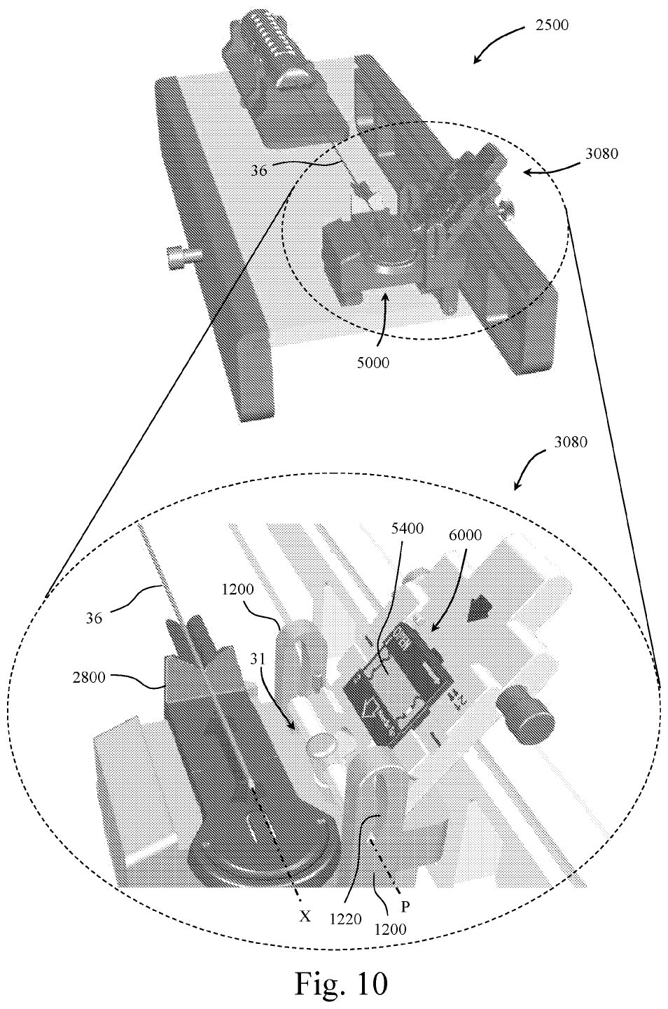

Attention is drawn to FIGS. 10 to 15 illustrating devices for collecting biological samples in accordance with various embodiments of the present invention. The embodiments discussed with respect to these figures may be considered being generally similar to at least some of those shown in former figures, such as in FIGS. 6A and 6B, in the sense of being suited for collecting a sample from a biopsy needle by performing a general rolling maneuver upon a biological sample within a biopsy needle's notch in order to collect it.

FIG. 10 schematically shows an embodiment of a device 2500 for collecting a biological sample initially carried on a surface of a beam—e.g. on a notch floor (not shown in this Figure) of a biopsy needle 36—onto a cassette 6000 having an adhering surface 5400. As better seen in the enlarged section at the lower side of the figure, cassette 6000 is detachably attached to the cassette holder 3080.

Biopsy needle 36 may be supported on a needle bed 2800, which in turn may be supported by a pedestal 5000 of the device. The collecting of the sample in this embodiment may be accomplished by displacing the cassette holder and cassette, while the biopsy gun remains static. In device 2500, a general rolling maneuver of the cassette holder 3080 relative to the biopsy needle 36 of the biopsy gun may be performed. The rolling maneuver may be accomplished as seen in this example by providing two side walls 1200 that project up from the pedestal 5000.

Each side wall may be formed with a groove 1220 forming an arched track and the cassette holder 3080 may comprise a hinge member 31 having a pivot axis P and pins coaxial with axis P and at opposing axial sides of the hinge member, which are configured each to slide inside a respective one of the grooves 1220 along the arched tracks. Axis P is generally parallel to the longitudinal axis X of the needle when the biopsy gun is supported in the device, and axis P may be arranged to remain generally parallel to axis X as the cassette holder is moved up or down along the arched tracks defined by the grooves 1220.

Thus, the rolling of the cassette holder 308 relative to biopsy needle 36 may be accomplished by combined movements of rotation of the cassette holder 308 about pivot axis P and by sliding of the cassette holder 308 up or down along the arched tracks through engagement of the pins within their respective grooves 1220.

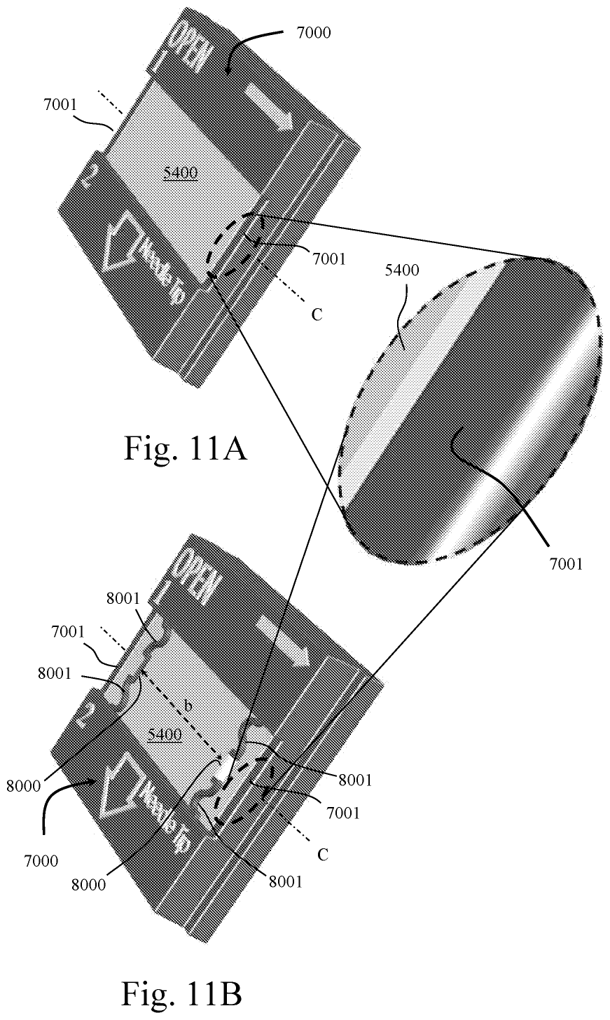

Attention is drawn to FIGS. 11A and 11B for closer views of various embodiments of cassettes in accordance with the present disclosure. Both cassettes as seen in these examples are formed with an adhering surface 5400 having an elongated rectangular shape that extends along a longitudinal axis C. The adhering surface in each one of the shown cassettes may be bound within a frame 7000, which includes shoulders 7001 that are located at opposing axial sides of the adhering surface.

As seen in the enlarged section common to both FIGS. 11A and 11B, an upper face of the adhering surface 5400 may be slightly lifted above the shoulders 7001 of the frame, in order to increase likelihood (as will be further noted in the disclosure herein below) that the adhering surface 5400 will meet the biopsy needle prior to the shoulders 7001.

The embodiment of the cassette seen in FIG. 11B can be seen being formed with a pair of blades 8000 resiliently coupled to the frame via resilient arms 8001. The blades 8000 as seen may be formed at generally opposing axial sides of the adhering surface, and axially spaced apart by a distance ‘b’ (that may be preferably measured between the opposing cutting edges of the opposing blades).

It is noted that the term blade as used herein refers to any part that can be used for clearing, wiping, scraping—portions of a biological sample that may be adhered to a biopsy needle and/or to regions of such needles at or adjacent a notch where a biological sample may typically be deposited.

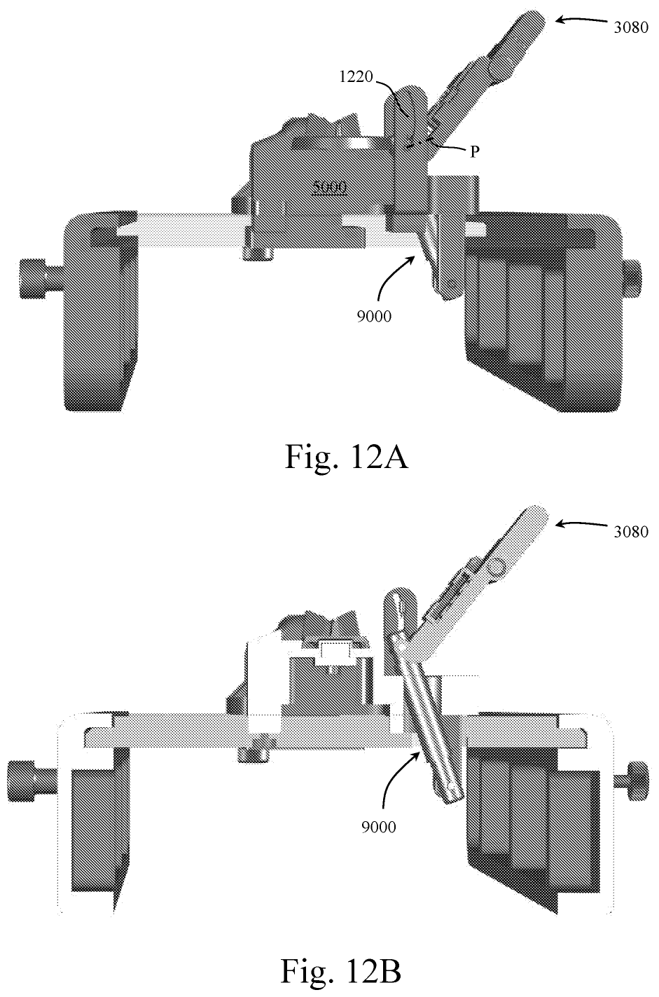

Attention is drawn to FIGS. 12A and 12B revealing a biasing member 9000 of the device that may be fixed at its lower side to pedestal 5000 and at its upper side to the cassette holder 3080 possibly to the hinge member 31 of the cassette holder. Biasing member 9000 may be arranged to apply a generally downward directed biasing force upon the cassette holder 3080 (possibly at the hinge member 31) in order to urge the pins of the hinge member 31 towards the lowermost end of the arched tracks of grooves 1220.

In an embodiment, when no other force is applied upon the cassette holder 3080, biasing member 9000 may be arranged to urge the hinge member with its pins to be at the lowermost ends of the respective arched tracks of grooves 1220.

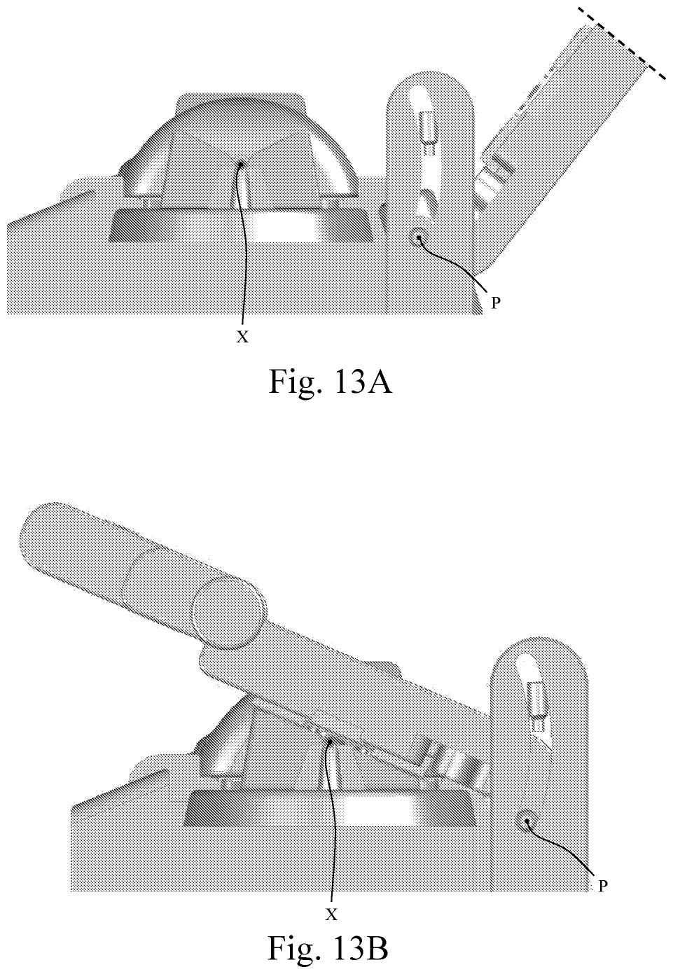

Attention is drawn to FIGS. 13A and 13B schematically showing cassette holder 3080 and of an axial tip of needle 36—as viewed along axes X and P of the needle and hinge member, respectively.

In FIG. 13A the cassette holder can be seen in an open state. A user operating the device to collect a sample located within the notch of the needle may initially urge the cassette holder to rotate about pivot axis P to the position seen in FIG. 13B, where the cassette holder's cassette at its adhering surface engages the needle. In these figures, the pins of the hinge member and hence axis P can be seen being maintained at the lowermost side of the arched track of each groove 1220, a state that can be assisted by the downward biasing force applied by biasing member 9000.

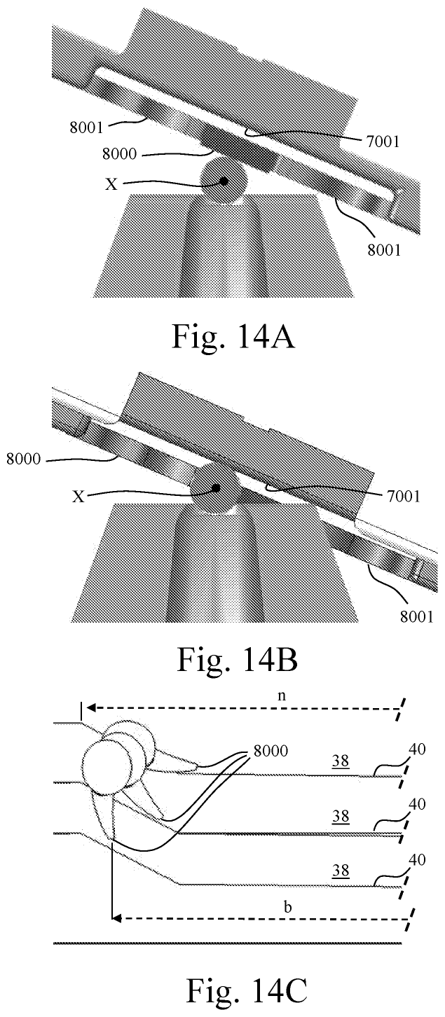

Attention is drawn to FIGS. 14A and 14B for closer views of possible stages of engagement between the cassette holder's cassette and needle in accordance with certain embodiments of the invention.

In embodiments, such as the one seen in FIG. 11B, where blades (e.g. 8000) are provided above the adhering surface, an initial engagement between the cassette and the needle may include (as seen in FIG. 14A) engagement between the blades and the needle. As seen in FIG. 14B, further revolving cassette holder about axis P may urge the blades to enter into the axial ends of the needle's notch until the shoulders 7001, which axially bound the adhering surface, engage the needle at axial locations outside of the notch.

In embodiments, such as the one seen in FIG. 11A, where blades are not provided above the adhering surface, the position of the cassette seen in FIG. 14B may represent the mutual relationship between the cassette and needle when the cassette is in the position seen in FIG. 13B.

That is to say, that revolving the cassette towards the needle about axis P may simply pass through the position seen in FIG. 14A with no distinct engagement action occurring there between the needle and cassette, until an end position is reached where the shoulders 7001 reach engagement with the needle at axial locations outside of the notch as seen in FIG. 14B.

It is noted that in embodiments where the adhering surface 5400 is arranged to be slightly lifted above the shoulders 7001 of the frame (as seen in the enlarged section common to both FIGS. 11A and 11B), higher likelihood exists that the adhering surface will slightly protrude into the notch and/or bear against the biological sample within the notch, and by that better adhere to the sample in order to collect it from the notch-when the cassette engages the needle (as seen in FIGS. 13A and 14B).

In an aspect of the present invention, provision of blades above the adhering surface (as e.g. seen in FIG. 11B), may assist detaching the biological sample from axial end regions of the notch in order to collect it from the needle. In the embodiment shown in FIG. 11B, where the blades are axially spaced apart, the assisting in detachment from the notch may accordingly occur from axial end regions of the notch.

Attention is drawn to FIG. 14C showing possible interactions between a blade 8000 and an axial end of a notch 38 having an axial extension ‘n’. In this figure, the same notch 38 is shown three times during three different stages of engagement between the blade 8000 and an axial end of the notch, with the lower notch representing an initial stage of engagement and the notches above the progressions of engagement between the blade and the notch.

In certain embodiments, providing an axial distance ‘b’ between the cutting edges of the blades that is similar or preferably slightly smaller than an axial extension ‘n’ of the notch—may assist in ensuring that the blades engage an end region of the notch where the biological sample may typically be caught on the needle.

As seen in FIG. 14C, in certain embodiments where the blade may be resiliently fixed within the cassette via e.g. the resilient arms 8001, such resilient fixation may allow the blade to adjust its orientation as it is urged into the notch (e.g. from the position seen in FIG. 14A towards that in FIG. 14B) and by that better follow a contour of the floor 40 of the notch. This may in turn assist in wiping the sample from its adherence to e.g. the floor of the notch so that the sample may then be removed from the notch.

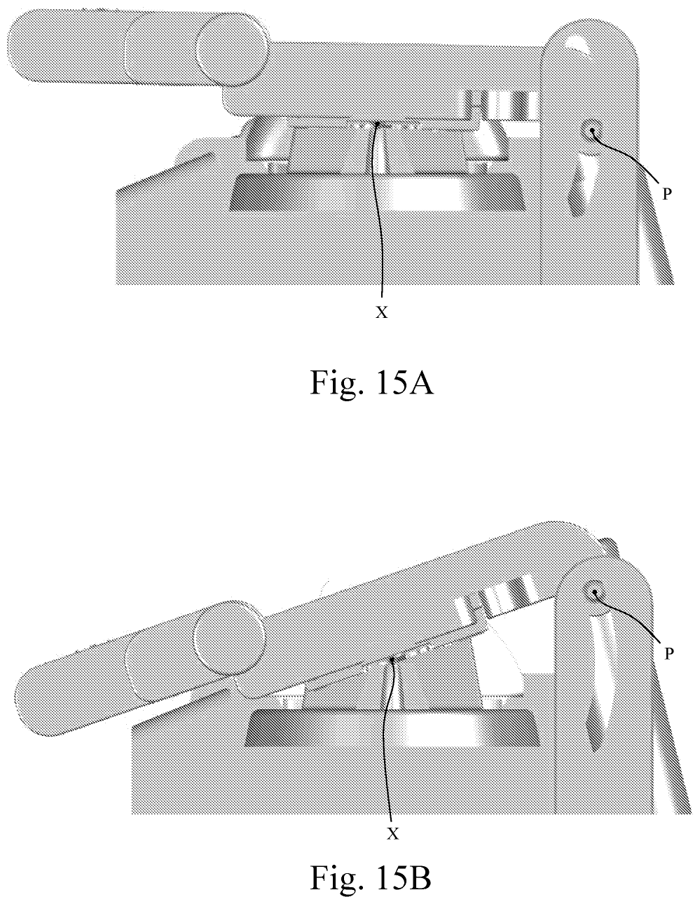

Attention is drawn to FIGS. 15A and 15B showing final stages of the rolling movement of the cassette upon the needle. Once engagement between the cassette and the needle (e.g. via interaction between the shoulders 7001 and the needle) has been reached as seen in FIG. 13B, further urging the cassette towards the needle is arranged to start rotating the cassette and cassette holder about axis X of the needle, while lifting the hinge member (with its axis P) up along the arched track defined by the grooves 1220 against the downward biasing force of the biasing member 9000.

This may continue until the pins of the hinge member reach their respective uppermost locations within their arched tracks as seen in FIG. 15B. Releasing the cassette holder in this state will urge it via the biasing member 9000 to return towards the open state of the cassette holder as seen e.g. in FIG. 12A and 12B, where access of a user to the cassette may allow removing the collected biological sample from the adhering surface of the cassette.

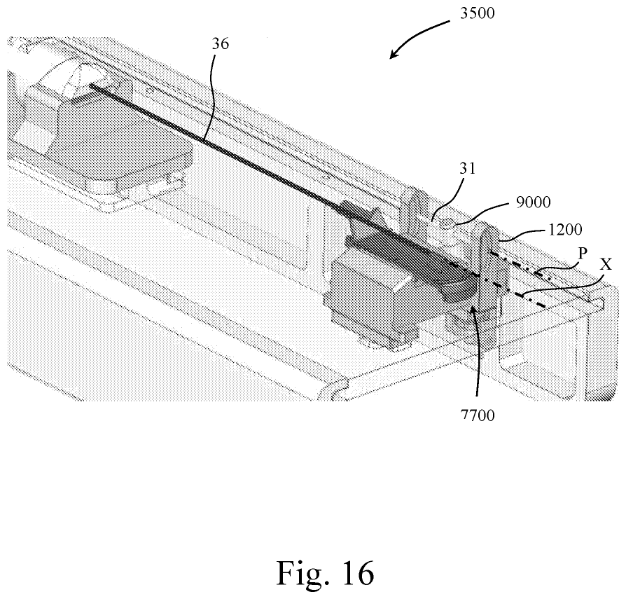

Attention is drawn to FIG. 16 schematically showing an embodiment of a device 3500 generally similar to the former embodiments previously shown and discussed (such as the one in FIG. 10). Device 3500 may be accordingly suited for collecting a biological sample initially carried on a surface of a beam—e.g. on a notch floor of a biopsy needle 36—onto a cassette having an adhering surface.

In this view elements of the device that are shown include the two side walls 1200 (comprising the grooves forming the arched tracks) together with the biasing member 9000 and the hinge member 31 with its axially extending pins (along axis P), while certain elements such as the cassette and its adhering surface are removed for the view for simplicity of discussion.

This embodiment reveals possible use of a cleaning crib 7700 for performing a preliminary cleaning action that may assist it detaching a biological sample from regions of the biopsy needle possibly outside of the notch. The cleaning crib in this example is embodied as a needle bed for supporting the axial forward end region of the biopsy needle 36.

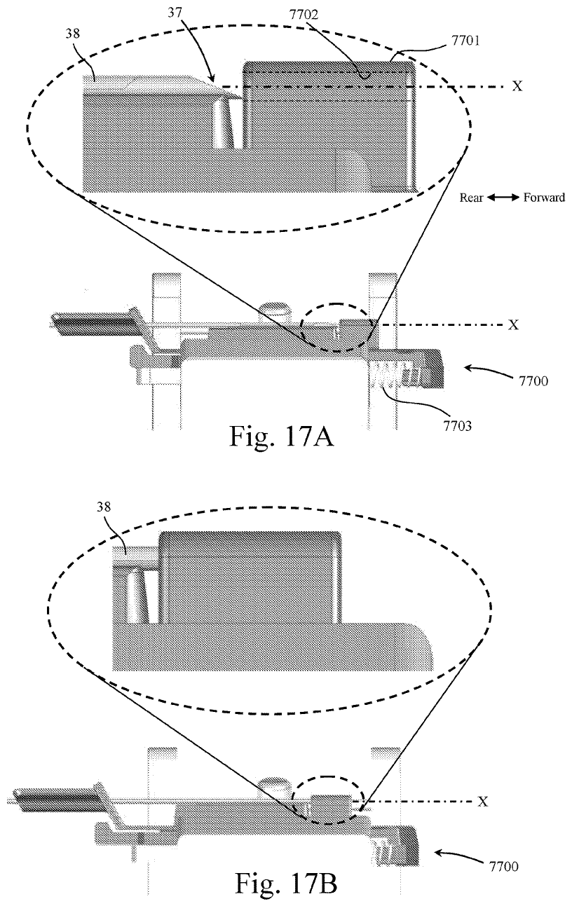

Attention is drawn to FIGS. 17A and 17B for closer side views of cleaning crib 7700. Cleaning crib 7700 may embodied as a movable element as seen in this example that is designed to substantially scrape the biological sample from the needle's periphery. The cleaning crib includes a rake member 7701, which in this example is formed with an axially extending internal passage 7702 that is coaxial with the longitudinal axis X of needle 36 and sized to allow needle to snuggly fit therein.

A resilient member 7703, in this example in the form of a spring, may be arranged to maintain rake member 7701 in a forward and an open state as seen in FIG. 17A. In the open state the forward axial tip 37 of the needle may be located rear to the rake member and axially outside of passage 7702.

Pressing the rake member 7701 in an axial rear direction against the resilient member may be arranged to urge the rake member with its passage move over the needle, while scraping any substances adjacent needle's axial tip 37 that may be stuck on the needle's periphery, such as portions of the biological sample that may be located outside of the needle's notch.

Preferably, the rake member 7701 may be urged in the axial rear direction against the resilient member to a position where at least a forward section of the needle's notch 38 may be located within passage 7702 as seen in FIG. 17B and its enlarged section.

Releasing rear pressure applied on the cleaning crib may allow the cleaning crib to move back in the forward direction towards the position seen in FIG. 17A, where the needle is again released from its position within passage 7702, and e.g. the process of collecting the biological sample from the needle as disclosed in the various device embodiments disclosed herein may start.

It is appreciated that certain aspects of some embodiments, which are, for clarity, described in the context of separate embodiments, may also be provided in combination in a single embodiment. Conversely, various aspects, which are, for brevity, described in the context of a single embodiment, may also be provided separately or in any suitable sub-combination or as suitable in any other described embodiment. Elements described in the context of an embodiment are not to be considered essential elements of that embodiment, unless the embodiment is inoperative without those elements.

Although the disclosure describes specific embodiments, alternatives, modifications and variations are apparent to those skilled in the art. Accordingly, the disclosure is intended to embrace all such alternatives, modifications and variations that fall within the scope of the appended claims.

Citation or identification of any reference in this application shall not be construed as an admission that such reference is available as prior art to the disclosure.

Section headings are used herein to ease understanding of the specification and should not be construed as necessarily limiting.

1-53. (canceled)

54. A device for collecting a biological sample from a biopsy needle, the device comprising:
a housing suitable for supporting a biopsy needle, and
a cassette comprising an adhering surface, wherein
the device being configured to collect the biological sample through motions comprising rotation of the cassette about a hinge member defining a pivot axis P and movement of the hinge member within the device.

55. The device of claim 54, wherein supporting of a needle is in a generally fixed position within the device.

56. The device of claim 55 and comprising a cassette holder for detachably housing the cassette therein.

57. The device of claim 56, wherein the cassette holder being coupled via the hinge member to a coupling position within the device, and the pivot axis P being configured to be generally parallel to a longitudinal axis X of a biopsy needle supported in the device

58. The device of claim 57, wherein the motion of movement of the hinge member is along a track formed in the device that extends transverse to the axial direction of the pivot axis P.

59. The device of claim 58 and comprising at least one groove defining the track, possibly an arched shaped track, and the moving of the hinge member and hence movement of the coupling position is along the track.

60. The device of claim 59, wherein the at least one groove is at least two grooves and the hinge member is coupled to the two grooves and is movable along the tracks defined by the grooves.

61. The device of claim 60, wherein each track extends between a beginning and an end, and the device comprising a biasing member for urging the hinge member towards the beginning of the track.

62. The device of claim 56, wherein the cassette comprises one or more blades located above its adhering surface for engaging a needle supported in the device prior to engagement of the adhering surface with same needle.

63. The device of claim 62, wherein the one or more blades being resiliently attached to the cassette.

64. A method for collecting a biological sample comprising the steps of:
providing a device comprising a housing and a cassette,
supporting a biopsy needle in a fixed position within the housing, and
urging motions within the device that comprise rotating the cassette about a hinge member that defines a pivot axis P and moving the hinge member within the device.

65. The method of claim 64, wherein the rotation about the hinge member urges an adhering surface of the cassette to engage with the biopsy needle, and moving the hinge member urges relative motion between the biopsy needle and the adhering surface in order to assist in collecting the biological sample from the biopsy needle.

66. The method of claim 65 and comprising a cassette holder for detachably housing the cassette therein, wherein the cassette holder being coupled via the hinge member to a coupling position within the device, and the pivot axis P being configured to be generally parallel to a longitudinal axis X of the biopsy needle.

67. The method of claim 66, wherein the collecting of the biological sample comprises first rotation about the hinge member and after that movement of the hinge member.

68. The method of claim 65, wherein the motion of movement of the hinge member comprises urging rolling of the adhering surface upon the needle.

69. The method of claim 68, wherein the motion of movement of the hinge member is along a track formed in the device that extends transverse to the axial direction of the pivot axis P.

70. The method of claim 69, wherein the device comprises at least one groove defining the track, possibly an arched shaped track, and the moving of the hinge member and hence movement of the coupling position is along the track.

71. The method of claim 69, wherein the at least one groove is at least two grooves and the hinge member is coupled to the two grooves and is movable along the tracks defined by the grooves.

72. The method of claim 69, wherein each track extends between a beginning and an end, and the device comprising a biasing member for urging the hinge member towards the beginning of the track.

73. The device of claim 69 and comprising one or more blades located above the adhering surface for engaging the needle prior to engagement of the adhering surface with the needle supported in the device.
